(19) United States
(12) Patent Application Publication
(10) Pub. No.: US 2008/0004263 A1
(43) Pub. Date: Jan. 3, 2008

(54) Ligands of Follicle Stimulating Hormone Receptor and Methods of Use Thereof

(76) Inventors: Vincent J. Santora, Carlsbad, CA (US); Jonathan A. Covel, San Diego, CA (US); Rena Hayashi, San Diego, CA (US); Robert R. Webb, San Diego, CA (US)

Correspondence Address: FISH & RICHARDSON P.C., P.O. BOX 1022, MINNEAPOLIS, MN, 55440-1022, US

(21) Appl. No.: 10/591,061

(22) PCT Filed: Mar. 3, 2005
(86) PCT No.: PCT/US05/07017

§ 371 Date: Jul. 3, 2007

Related U.S. Application Data
(60) Provisional application No. 60/550,161, filed on Mar. 4, 2004.

(51) Int. Cl. A61K 31/4545; A61K 31/454; A61K 31/5377; A61K 31/541; A61P 15/10; C07D 211/82; C07D 221/00; C07D 279/12; C07D 413/14; C12N 5/02

(52) U.S. Cl. 514/227.8; 435/375; 514/236.8; 514/326; 544/130; 544/58.2; 546/194; 546/209

(57) ABSTRACT

The present invention relates to compounds Formula I (I) that are modulators of the follicle stimulating hormone (FSH) receptor and are useful in the treatment of infertility and related disorders.

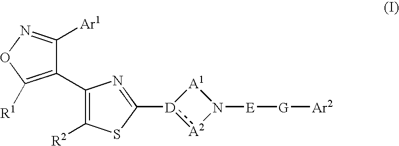

### FIELD OF THE INVENTION

The present invention relates to small molecule modulators of the follicle stimulating hormone (FSH) receptor that are useful, for example, in the treatment of fertility disorders.

### BACKGROUND OF THE INVENTION

Infertility is a widespread problem affecting millions of couples worldwide. Follicle stimulating hormone, a pituitary-derived heterodimeric glycoprotein hormone, has been used in the medical field for treating fertility disorders by, for example, inducing ovulation and/or controlling ovarial hyperstimulation. FSH is typically obtained by either extraction from urine or produced by recombinant DNA technology. FSH has been used in a clinical setting for more than 40 years and remains one of the most effective compounds on the market for the treatment of infertility.

FSH binds to and activates the FSH receptor which is known to be a member of the family of G protein-coupled receptors (GPCRs). When activated, these receptors stimulate an increase in the activity of adenylyl cyclase, which results in an increase in the level of the intracellular second messenger adenosine 3′,5′-monophosphate (cAMP), which in turn causes increased steroid synthesis and secretion.

FSH receptor has been found to be expressed in the tissues of both male and female mammals. In females, FSH stimulates follicular granulosa cell proliferation in the ovary and impacts synthesis of estrogen, a hormone involved in follicular maturation and ovulation. In males, FSH is involved in the maturation of sperm cells. More particularly, FHS acts in the testicular Sertoli cells which support spermatogenesis. Accordingly, ligand modulators of the FSH receptor can potentially be useful in the treatment of a number of fertility disorders including infertility in both men and women.

While FSH has been widely used in connection with a number of fertility-related treatments or procedures, it remains expensive and is difficult to administer because it cannot be delivered orally. Accordingly, the identification of new small molecule ligands of the FSH receptor is desirable for the development of improved therapies for infertility and other disorders. The compounds, compositions and methods described herein help fulfill these and other needs.

### SUMMARY OF THE INVENTION

The present invention provides, inter alia, compounds of Formula I: 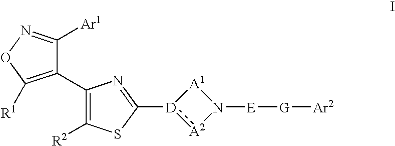
wherein constituent members are defined herein, that are modulators of the FSH receptor.

The present invention further provides a composition comprising a compound of Formula I and a pharmaceutically acceptable carrier.

The present invention further provides a method of modulating (e.g., activating) the follicle stimulating hormone (FSH) receptor comprising contacting said receptor with a compound of Formula I.

The present invention further provides a method of increasing adenylyl cyclase activity or the level of 5′-monophosphate (cAMP) in a cell, cell culture or tissue expressing the follicle stimulating hormone receptor comprising contacting the cell, cell culture or tissue with a compound of Formula I.

The present invention further provides a method of inducing ovulation in a female mammal comprising administering to the female mammal an ovulation-inducing amount of a compound of Formula I.

The present invention further provides a method of treating a fertility disorder in a patient comprising administering to the patient a therapeutically effective amount of a compound of Formula I.

The present invention further provides a method of treating infertility in a female patient comprising administering to said female patient a therapeutically effective amount of a compound of Formula I.

### DETAILED DESCRIPTION

The present invention is drawn to compounds, which bind to and modulate the activity of a GPCR referred to herein as FSH receptor, and uses thereof. The term FSH receptor, as used herein, includes the human sequence found in GenBank accession number AAA52477, naturally-occurring allelic variants, mammalian orthologs, biologically active fragments and recombinant mutants thereof.

The present invention provides, inter alia, a compound of Formula I: 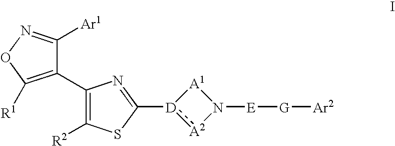
or a pharmaceutically acceptable salt, hydrate or solvate thereof, wherein:

Ar¹ is aryl, heteroaryl, biaryl, biheteroaryl, arylheteroaryl or heteroarylaryl, wherein Ar¹ is optionally substituted with one or more substituents selected from halo, cyano, nitro, C₁₋₆ alkyl, C₁₋₆ haloalkyl, C₂₋₆ alkenyl, C₂₋₆ alkynyl, carbocyclyl optionally substituted by one or more R¹³, heterocyclyl optionally substituted by one or more R¹³, carbocyclylalkyl optionally substituted by one or more R¹³, carbocyclylalkenyl optionally substituted by one or more R¹³, carbocyclylalkynyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸;

Ar² is aryl or heteroaryl, each optionally substituted with one or more substituents selected from halo, cyano, nitro, C₁₋₆ alkyl, C₁₋₆ haloalkyl, C₂₋₆ alkenyl, C₂₋₆ alkynyl, carbocyclyl optionally substituted by one or more R¹⁴, heterocyclyl optionally substituted by one or more R¹⁴, hydroxylamino, OR⁹, SR⁹, SOR¹⁰, SO₂R¹⁰, COR¹⁰, COOR⁹, OC(O)R¹⁰ or NR¹¹R¹²;

D is N, C or CR³;

[symbol] is a single bond when D is N or CR³;

[symbol] is a double bond when D is C;

A¹ is absent or a C₁₋₃ straight-chain aliphatic group optionally substituted with one or more substituents selected from halo, C₁₋₄ alkyl, C₁₋₄ haloalkyl, C₁₋₄ alkoxy, C₁₋₄ haloalkoxy, amino, (C₁₋₆ alkyl)amino, di(C₁₋₆ alkyl)amino, hydroxy, carboxy, (C₁₋₄ alkoxy)carbonyl, or cyano;

A² is C₁₋₄ straight-chain aliphatic group optionally substituted with one or more substituents selected from halo, C₁₋₄ alkyl, C₁₋₄ haloalkyl, C₁₋₄ alkoxy, C₁₋₄ haloalkoxy, amino, (C₁₋₆alkyl)amino, di(C₁₋₆ alkyl)amino, hydroxy, carboxy, (C₁₋₄ alkoxy)carbonyl, or cyano;

E is CO, C(O)O, C(O)NR⁴, NR⁴CONR⁴, SO, SO₂, SONR⁴, SO₂NR⁴, or a bond;

G is C₁₋₃ alkylene, C₂₋₃ alkenylene or C₂₋₃ alkynylene optionally substituted with one or more substituents selected from halo, C₁₋₄ alkyl, C₁₋₄ haloalkyl, C₁₋₄ alkoxy, C₁₋₄ haloalkoxy, amino, (C₁₋₄ alkyl)amino, di(C₁₋₄ alkyl)amino, hydroxy, carboxy, (C₁₋₄ alkoxy)carbonyl, or cyano;

R¹ is H, C₁₋₆ alkyl, C₂₋₆ alkenyl or C₂₋₆ alkynyl, wherein R¹ is optionally substituted with one or more substituents selected from halo, OH, SH, nitro, cyano, C₁₋₄ haloalkyl, C₁₋₅ acyl, C₁₋₅ acyloxy, C₁₋₄ alkoxy, C₁₋₄ thioalkoxy, C₁₋₄ haloalkoxy, amino, (C₁₋₄ alkyl)amino, di(C₁₋₄ alkyl)amino, aminocarbonyl, (C₁₋₄ alkyl)aminocarbonyl, di(C₁₋₄ alkyl)aminocarbonyl, C₁₋₄ alkylsulfinyl, C₁₋₄ alkylsulfonyl, C₁₋₄ haloalkylsulfinyl, C₁₋₄ haloalkylsulfonyl, aminosulfonyl, (C₁₋₄ alkyl)aminosulfonyl, di(C₁₋₄ alkyl)aminosulfonyl, ureido, C₁₋₄ alkylureido, di(C₁₋₄ alkyl)ureido, thioureido, C₁₋₄ alkylthioureido, di(C₁₋₄ alkyl)thioureido, carboxy, (C₁₋₆ alkoxy)carbonyl, and hydroxylamino;

R² is H, C₁₋₆ alkyl, C₂₋₆ alkenyl or C₂₋₆ alkynyl, wherein R² is optionally substituted with one or more substituents selected from halo, OH, SH, nitro, cyano, C₁₋₄ haloalkyl, C₁₋₅ acyl, C₁₋₅ acyloxy, C₁₋₄ alkoxy, C₁₋₄ thioalkoxy, C₁₋₄ haloalkoxy, amino, (C₁₋₄ alkyl)amino, di(C₁₋₄ alkyl)amino, aminocarbonyl, (C₁₋₄ alkyl)aminocarbonyl, di(C₁₋₄ alkyl)aminocarbonyl, C₁₋₄ alkylsulfinyl, C₁₋₄ alkylsulfonyl, C₁₋₄ haloalkylsulfinyl, C₁₋₄ haloalkylsulfonyl, aminosulfonyl, (C₁₋₄ alkyl)aminosulfonyl, di(C₁₋₄ alkyl)aminosulfonyl, ureido, C₁₋₄ alkylureido, di(C₁₋₄ alkyl)ureido, thioureido, C₁₋₄ alkylthioureido, di(C₁₋₄ alkyl)thioureido, carboxy, (C₁₋₆ alkoxy)carbonyl, and hydroxylamino;

or R¹ and R² together with the carbon atoms to which they are attached and the two carbon atoms through which the isoxazole and thiazole moieties of the core are joined form a fused C₅₋₇ carbocyclyl group or fused 5-7 membered heterocyclyl group optionally substituted with one or more substituents selected from halo, C₁₋₄ alkyl, C₁₋₄ haloalkyl, C₁₋₄ alkoxy, C₁₋₄ haloalkoxy, amino, (C₁₋₄ alkyl)amino, di(C₁₋₄ alkyl)amino, hydroxy, carboxy, (C₁₋₄ alkoxy)carbonyl, or cyano;

R³ is H or C₁₋₆ alkyl;

R⁴, at each independent occurrence, is H or C₁₋₄ alkyl;

R⁵ and R⁹ are each, independently, H, C₁₋₈ alkyl, C₁₋₈ haloalkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl or (5-7 membered heterocycloalkyl)alkyl;

R⁶ and R¹⁰ are each, independently, H, C₁₋₈ alkyl, C₁₋₈ haloalkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, amino, (C₁₋₄ alkyl)amino, di(C₁₋₄ alkyl)amino,

R⁷ and R⁸ are each, independently, H, C₁₋₈ alkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, (C₁₋₈ alkyl)carbonyl, (C₁₋₈ haloalkyl)carbonyl, (C₁₋₈ alkoxy)carbonyl, (C₁₋₈ haloalkoxy)carbonyl, (C₁₋₄ alkyl)sulfonyl, (C₁₋₄ haloalkyl)sulfonyl or arylsulfonyl;

or R⁷ and R⁸, together with the N atom to which they are attached form a 5-7 membered heterocycloalkyl group;

R¹¹ and R¹² are each, independently, H, C₁₋₈ alkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, (C₁₋₈ alkyl)carbonyl, (C₁₋₈ haloalkyl)carbonyl, (C₁₋₈ alkoxy)carbonyl, (C₁₋₈ haloalkoxy)carbonyl, (C₁₋₄ alkyl)sulfonyl, (C₁₋₄ haloalkyl)sulfonyl or arylsulfonyl;

or R¹¹ and R¹², together with the N atom to which they are attached form a 5-7 membered heterocycloalkyl group; and

R¹³ and R¹⁴ are each, independently, halo, cyano, nitro, C₁₋₄ alkyl, C₁₋₄ haloalkyl, C₁₋₄ alkoxy, C₁₋₄ haloalkoxy, amino, (C₁₋₄ alkyl)amino, di(C₁₋₄ alkyl)amino, hydroxy, carboxy, (C₁₋₄ alkoxy)carbonyl, C₁₋₄ acyl, C₁₋₄ acyloxy, aminocarbonyl, (C₁₋₄ alkyl)aminocarbonyl, or di(C₁₋₄ alkyl)aminocarbonyl.

In some embodiments, Ar¹ is aryl, heteroaryl, biaryl, biheteroaryl, arylheteroaryl or heteroarylaryl, wherein Ar¹ is substituted with 1, 2 or 3 substituents selected from halo, cyano, nitro, C₁₋₆ alkyl, C₁₋₆ haloalkyl, C₂₋₆ alkenyl, C₂₋₆ alkynyl, carbocyclyl optionally substituted by one or more R¹³, heterocyclyl optionally substituted by one or more R¹³, carbocyclylalkyl optionally substituted by one or more R¹³, carbocyclylalkenyl optionally substituted by one or more R¹³, carbocyclylalkynyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸.

In some embodiments, Ar¹ is aryl, heteroaryl, biaryl, biheteroaryl, arylheteroaryl or heteroarylaryl, wherein Ar¹ is optionally substituted with one or more substituents selected from halo, cyano, nitro, heterocyclyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸.

In some embodiments, Ar¹ is aryl, biaryl or heteroarylaryl, wherein Ar¹ is optionally substituted with one or more substituents selected from halo, cyano, nitro, C₁₋₆ alkyl, C₁₋₆ haloalkyl, C₂₋₆ alkenyl, C₂₋₆ alkynyl, carbocyclyl optionally substituted by one or more R¹³, heterocyclyl optionally substituted by one or more R¹³, carbocyclylalkyl optionally substituted by one or more R¹³, carbocyclylalkenyl optionally substituted by one or more R¹³, carbocyclylalkynyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸.

In some embodiments, Ar¹ is phenyl, biphenyl or heteroarylphenyl, wherein Ar¹ is optionally substituted with one or more substituents selected from halo, cyano, nitro, C₁-C₆ alkyl, C₁-C₆ haloalkyl, C₂-C₆ alkenyl, C₂-C₆ alkynyl, carbocyclyl optionally substituted by one or more R¹³, heterocyclyl optionally substituted by one or more R¹³, carbocyclylalkyl optionally substituted by one or more R¹³, carbocyclylalkenyl optionally substituted by one or more R¹³, carbocyclylalkynyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸.

In some embodiments, Ar¹ is phenyl, biphenyl or heteroarylphenyl, wherein Ar¹ is optionally substituted with 1, 2 or 3 substituents selected from halo, cyano, nitro, heterocyclyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸.

In some embodiments, Ar¹ is phenyl, biphenyl or heteroarylphenyl, wherein Ar¹ is substituted with 1, 2 or 3 substituents selected from halo, cyano, nitro, heterocyclyl optionally substituted by one or more R¹³, heterocyclylalkynyl optionally substituted by one or more R¹³, C₁₋₄alkoxy, SO₂R⁶, COR⁶, COOR⁵ or NR⁷R⁸.

In some embodiments, Ar² is aryl or heteroaryl, each optionally substituted with 1, 2 or 3 substituents selected from halo, cyano, nitro, C₁₋₆ alkyl, C₁₋₆ haloalkyl, C₂₋₆ alkenyl, C₂₋₆ alkynyl, carbocyclyl optionally substituted by one or more R¹⁴, heterocyclyl optionally substituted by one or more R¹⁴, hydroxylamino, OR⁹, SR⁹, SOR¹⁰, SO₂R¹⁰, COR¹⁰, COOR⁹, OC(O)R¹⁰ or NR¹¹R¹².

In some embodiments, Ar² is aryl or heteroaryl.

In some embodiments, Ar² is heteroaryl.

In some embodiments, Ar² is thienyl.

In some embodiments, Ar² is aryl.

In some embodiments, Ar² is phenyl.

In some embodiments, D is CR³.

In some embodiments, D is CH.

In some embodiments, A¹ is a C₁₋₃ alkylene group.

In some embodiments, A¹ is CH₂ or CH₂CH₂.

In some embodiments, A¹ is absent.

In some embodiments, D is CR³ and A² is a C₁₋₃ alkylene group.

In some embodiments, D is CR³ and A² is CH₂CH₂ or CH₂CH₂CH₂.

In some embodiments, D is CR³, A¹ is CH₂CH₂, and A² is CH₂CH₂.

In some embodiments, D is CR³, A¹ is absent, and A² is CH₂CH₂CH₂.

In some embodiments, E is CO, C(O)O, C(O)NR⁴, SO₂ or a bond.

In some embodiments, E is CO or SO₂.

In some embodiments, E is CO.

In some embodiments, G is C₁₋₃ alkylene.

In some embodiments, G is CH₂ or CH₂CH₂.

In some embodiments, wherein G is CH₂.

In some embodiments, R¹ is H or C₁₋₄ alkyl.

In some embodiments, R¹ is methyl.

In some embodiments:

R¹ is H, C₁₋₆ alkyl, C₂₋₆ alkenyl or C₂₋₆ alkynyl, wherein R¹ is optionally substituted with one or more substituents selected from halo, OH, SH, nitro, cyano, C₁₋₄ haloalkyl, C₁₋₅ acyl, C₁₋₅ acyloxy, C₁₋₄ alkoxy, C₁₋₄ thioalkoxy, C₁₋₄ haloalkoxy, amino, (C₁₋₄ alkyl)amino, di(C₁₋₄ alkyl)amino, aminocarbonyl, (C₁₋₄ alkyl)aminocarbonyl, di(C₁₋₄ alkyl)aminocarbonyl, C₁₋₄ alkylsulfinyl, C₁₋₄ alkylsulfonyl, C₁₋₄ haloalkylsulfinyl, C₁₋₄ haloalkylsulfonyl, aminosulfonyl, (C₁₋₄ alkyl)aminosulfonyl, di(C₁₋₄ alkyl)aminosulfonyl, ureido, C₁₋₄ alkylureido, di(C₁₋₄ alkyl)ureido, thioureido, C₁₋₄ alkylthioureido, di(C₁₋₄ alkyl)thioureido, carboxy, (C₁₋₆ alkoxy)carbonyl, and hydroxylamino; and

R² is H, C₁₋₆ alkyl, C₂₋₆ alkenyl or C₂₋₆ alkynyl, wherein R² is optionally substituted with one or more substituents selected from halo, OH, SH, nitro, cyano, C₁₋₄ haloalkyl, C₁₋₅ acyl, C₁₋₅ acyloxy, C₁₋₄ alkoxy, C₁₋₄ thioalkoxy, C₁₋₄ haloalkoxy, amino, (C₁₋₄ alkyl)amino, di(C₁₋₄ alkyl)amino, aminocarbonyl, (C₁₋₄ alkyl)aminocarbonyl, di(C₁₋₄ alkyl)aminocarbonyl, C₁₋₄ alkylsulfinyl, C₁₋₄ alkylsulfonyl, C₁₋₄ haloalkylsulfinyl, C₁₋₄ haloalkylsulfonyl, aminosulfonyl, (C₁₋₄ alkyl)aminosulfonyl, di(C₁₋₄ alkyl)aminosulfonyl, ureido, C₁₋₄ alkylureido, di(C₁₋₄ alkyl)ureido, thioureido, C₁₋₄ alkylthioureido, di(C₁₋₄ alkyl)thioureido, carboxy, (C₁₋₆ alkoxy)carbonyl, and hydroxylamino.

In some embodiments, R² is H or C₁₋₄ alkyl.

In some embodiments, R² is H.

In some embodiments, R³ is H.

In some embodiments, R⁴, at each independent occurrence, is H.

In some embodiments:

D is CR³;

A¹ is a absent or a C₁₋₃ alkylene group;

A² is a C₁₋₃ alkylene group;

E is CO, C(O)O, C(O)NR⁴, SO₂ or a bond;

G is C₁₋₃ alkylene;

R¹ is H or C₁₋₆ alkyl; and

R² is H or C₁₋₆ alkyl.

In some embodiments:

Ar² is aryl or heteroaryl, each optionally substituted with 1, 2 or 3 substituents selected from halo, cyano, nitro, C₁-C₆ alkyl, C₁-C₆ haloalkyl, C₂-C₆ alkenyl, C₂-C₆ alkynyl, carbocyclyl optionally substituted by one or more R¹⁴, heterocyclyl optionally substituted by one or more R¹⁴, hydroxylamino, OR⁹, SR⁹, SOR¹⁰, SO₂R¹⁰, COR¹⁰, COOR⁹, OC(O)R¹⁰ or NR¹¹R¹²;

D is CR³;

A¹ is absent or a C₁₋₃ alkylene group;

A² is a C₁₋₃ alkylene group;

E is CO, C(O)O, C(O)NR⁴, SO₂ or a bond;

G is C₁₋₃ alkylene;

R¹ is H or C₁₋₆ alkyl; and

R² is H or C₁₋₆ alkyl.

In some embodiments:

Ar¹ is phenyl, biphenyl or heteroarylphenyl, wherein Ar¹ is optionally substituted with one or more substituents selected from halo, cyano, nitro, C₁-C₆ alkyl, C₁-C₆ haloalkyl, C₂-C₆ alkenyl, C₂-C₆ alkynyl, carbocyclyl optionally substituted by one or more R¹³, heterocyclyl optionally substituted by one or more R¹³, carbocyclylalkyl optionally substituted by one or more R¹³, carbocyclylalkenyl optionally substituted by one or more R¹³, carbocyclylalkynyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸;

Ar² is aryl or heteroaryl;

D is CR³;

A¹ is absent or a C₁₋₃ alkylene group;

A² is a C₁₋₃ alkylene group;

E is CO, C(O)O, C(O)NR⁴, SO₂ or a bond;

G is C₁₋₃ alkylene;

R¹ is H or C₁₋₆ alkyl; and

R² is H or C₁₋₆ alkyl.

In some embodiments:

Ar¹ is phenyl, biphenyl or heteroarylphenyl, wherein Ar¹ is optionally substituted with 1, 2 or 3 substituents selected from halo, cyano, nitro, heterocyclyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸;

Ar² is aryl or heteroaryl;

D is CR³;

A¹ is absent or a C₁₋₃ alkylene group;

A² is a C₁₋₃ allylene group;

E is CO, C(O)O, C(O)NR⁴, SO₂ or a bond;

G is C₁₋₃ alkylene;

R¹ is H or C₁₋₆ alkyl; and

R² is H or C₁₋₆ alkyl.

In some embodiments:

Ar¹ is phenyl, biphenyl or heteroarylphenyl, wherein Ar¹ is optionally substituted with 1, 2 or 3 substituents selected from halo, cyano, nitro, heterocyclyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸;

Ar² is aryl or heteroaryl;

D is CH;

A¹ is absent, CH₂ or CH₂CH₂;

A² is CH₂CH₂ or CH₂CH₂CH₂;

E is CO, SO₂ or a bond;

G is CH₂ or CH₂CH₂;

R¹ is C₁₋₄ alkyl; and

R² is H.

In some embodiments:

Ar¹ is phenyl, biphenyl or heteroarylphenyl, wherein Ar¹ is substituted with 1, 2 or 3 substituents selected from halo, cyano, nitro, heterocyclyl optionally substituted by one or more R¹³, heterocyclylalkynyl optionally substituted by one or more R¹³, C₁₋₄ alkoxy, SO₂R⁶, COR⁶, COOR⁵ or NR⁷R⁸;

Ar² is aryl or heteroaryl;

D is CH;

A¹ is absent, CH₂ or CH₂CH₂;

A² is CH₂CH₂ or CH₂CH₂CH₂;

E is CO, SO₂ or a bond;

G is CH₂ or CH₂CH₂;

R¹ is C₁₋₄ alkyl; and

R² is H.

It is appreciated that certain features of the invention, which are, for clarity, described in the context of separate embodiments, can also be provided in combination in a single embodiment. Conversely, various features of the invention which are, for brevity, described in the context of a single embodiment, may also be provided separately or in any suitable subcombination.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, the term “alkylene” refers to a bivalent alkyl moiety.

As used herein, “alkenyl” refers to an alkyl group having one or more double carbon-carbon bonds. Example alkenyl groups include ethenyl, propenyl, butenyl, pentenyl, hexenyl, butadienyl, pentadienyl, hexadienyl, and the like.

As used herein, “alkynyl” refers to an alkyl group having one or more triple carbon-carbon bonds. Example alkynyl groups include ethynyl, propynyl, butynyl, pentynyl, and the like.

As used herein, “aliphatic group” refers to any non-cyclic hydrocarbon, including alkyl, alkenyl, and alkynyl groups. Aliphatic groups can be straight-chain or branched as well as monovalent or bivalent (e.g., linking). Example bivalent aliphatic groups include methylene (CH₂), ethylene (CH₂CH₂), propylene (CH₂CH₂CH₂), ethenylene (CH═CH), propenylene (CH₂—CH═CH), ethynylene (C≡C), propynylene (CH₂—C≡C), and the like. In some embodiments, an aliphatic group has 1 to about 10 carbon atoms, 1 to about 5 carbon atoms, 1 to about 3 carbon atoms, or 2 to about 3 carbon atoms.

As used herein, “haloalkyl” refers to an alkyl group having one or more halogen substituents. Example haloalkyl groups include CF₃, C₂F₅, CHF₂, CCl₃, CHCl₂, C₂Cl₅, and the like. An alkyl group in which all of the hydrogen atoms are replaced with halogen atoms can be referred to as “perhaloalkyl.” Example perhaloalkyl groups include CF₃ and C₂F₅.

As used herein, “carbocyclyl” refers to groups that are saturated (i.e., containing no double or triple bonds) or unsaturated (i.e., containing one or more double or triple bonds) cyclic hydrocarbon moieties. Carbocyclyl groups can be mono- or polycyclic. Example carbocyclyl groups include cyclopropyl, cyclobutyl, cyclopentyl, cyclohexyl, cycloheptyl, cyclopentenyl, 1,3-cyclopentadienyl, cyclohexenyl, norbornyl, norpinyl, norcarnyl, adamantyl, phenyl, and the like. Carbocyclyl groups can be aromatic (e.g., “aryl”) or non-aromatic (e.g., “cycloalkyl”). In some embodiments, carbocyclyl groups can have from 3 to about 20, 3 to about 10, or 3 to about 7 carbon atoms.

As used herein, “aryl” refers to monocyclic or polycyclic aromatic hydrocarbons such as, for example, phenyl, naphthyl, anthracenyl, phenanthrenyl, indanyl, indenyl, and the like. In some embodiments, aryl groups have from 6 to about 18 carbon atoms.

As used herein, “biaryl” refers an aryl group substituted by a further aryl group. The two aryl groups can be the same or different. An example biaryl group is biphenyl.

As used herein, “cycloalkyl” refers to non-aromatic cyclic hydrocarbons, including cyclized alkyl, alkenyl, and alkynyl groups. Cycloalkyl group can include bi- or poly-cyclic ring systems and can optionally contain unsaturations. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo derivatives of cyclopentane (indanyl), cyclohexane (tetrahydronaphthyl), and the like. Cycloalkyl groups can have from about 3 to about 20, 3 to about 12, or 3 to about 7 carbon atoms.

As used herein, “heterocyclyl” refers to a group that can be a saturated or unsaturated carbocyclyl group wherein one or more of the ring-forming carbon atoms of the carbocyclyl group is replaced by a heteroatom such as O, S, or N. Heterocyclyl groups can be aromatic (e.g., “heteroaryl”) or non-aromatic (e.g., “heterocycloalkyl”). Heterocyclyl groups can correspond to hydrogenated and partially hydrogenated heteroaryl groups. Heterocarbocyclyl groups can contain, in addition to at least one heteroatom, from about 1 to about 20, about 2 to about 10, or about 2 to about 7 carbon atoms and can be attached through a carbon atom or heteroatom. In some embodiments, heterocyclyl groups can have from 3 to 20, 3 to 10, 3 to 7, or 5 to 7 ring-forming atoms. Further, heterocyclyl groups can be substituted or unsubstituted. Examples of heterocyclyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like as well as any of the groups listed for heteroaryl and heterocycloalkyl.

As used herein, “heteroaryl” groups are monocyclic and polycyclic aromatic hydrocarbons that have at least one heteroatom ring member such as sulfur, oxygen, or nitrogen. Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrrolyl, oxazolyl, benzofuryl, benzothienyl, benzthiazolyl, isoxazolyl, pyrazolyl, triazolyl, tetrazolyl indazolyl, 1,2,4-thiadiazolyl, isothiazolyl, benzothienyl, purinyl, carbazolyl, benzimidazolyl, 2,3-dihydrobenzofuranyl, 2,3-dihydrobenzothienyl, 2,3-dihydrobenzothienyl-5-oxide, 2,3-dihydrobenzothienyl-5-dioxide, benzoxazolin-2-on-yl, indolinyl, benzodioxolanyl, benzodioxane, and the like. In some embodiments, heteroaryl groups can have from 1 to about 20 carbon atoms, and in further embodiments from about 3 to about 20 carbon atoms. In some embodiments, heteroaryl groups have 1 to about 4, 1 to about 3, or 1 to 2 heteroatoms.

As used herein, “biheteroaryl” refers to a heteroaryl group substituted by a further heteroaryl group. The two heteroaryl groups can be the same or different. An example of a biheteroaryl group is bipyridyl.

As used herein, “heterocycloalkyl” refers to a non-aromatic hydrocarbon including cyclized alkyl, alkenyl, and alkynyl groups where one or more of the ring-forming carbon atoms is replaced by a heteroatom such as an O, N, or S atom. Also included in the definition of heterocycloalkyl are moieties that have one or more aromatic rings fused (i.e., having a bond in common with) to the non-aromatic heterocyclic ring, for example phthalimidyl, naphthalimidyl pyromellitic diimidyl, phthalanyl, and benzo derivatives of saturated heterocycles such as indolene and isoindolene groups.

As used herein, “arylheteroaryl” refers to a heteroaryl group substituted by an aryl group.

As used herein, “heteroarylaryl” refers to an aryl group substituted by a heteroaryl group. An example heteroarylaryl group is heteroarylphenyl which is a phenyl group substituted by a heteroaryl group.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like. “Haloalkoxy” refers to an —O-haloalkyl group.

As used herein, “alkoxycarbonyl” refers to a carbonyl group substituted by an alkoxy group.

As used herein, “thioalkoxy” refers to an —S-alkyl group.

As used herein, “carbocyclylalkyl” refers to an alkyl group substituted by a carbocyclyl group. An example “carbocyclylalkyl” group is an “aralkyl” or “arylalkyl” group which corresponds to an alkyl moiety substituted by an aryl group. Example aralkyl groups include phenylalkyl groups such as benzyl, phenethyl (1-phenylethyl or 2-phenylethyl), phenpropyl, naphthylmethyl, and the like. Another example “carbocyclylalkyl” group is a “cycloalkylalkyl” group which corresponds to an alkyl group substituted by a cycloalkyl group. An example cycloalkylalkyl group is cyclopropylmethyl.

As used herein, “carbocyclylalkenyl” refers to an alkenyl group substituted by a carbocyclyl group.

As used herein, “carbocyclylalkynyl” refers to an alkynyl group substituted by a carbocyclyl group.

As used herein, the term “heterocyclylalkyl” refers to an alkyl group substituted by a heterocyclyl group. An example of a “heterocyclylalkyl” group is a “heteroarylalkyl” group which refers to an alkyl moiety substituted by a heteroaryl moiety. Another example “heterocyclylalkyl” group is a “heterocycloalkylalkyl” group which corresponds to an alkyl group substituted by heterocycloalkyl group.

As used herein, “heterocyclylalkenyl” refers to an alkenyl group substituted by a heterocyclyl group.

As used herein, “heterocyclylalkynyl” refers to an alkynyl group substituted by a heterocyclyl group.

As used herein, the term “amino” refers to NH₂. Similarly, the term “alkylamino” refers to an amino group substituted by an alkyl group, and the term “dialkylamino” refers to an amino group substituted by two alkyl groups.

As used herein, the term “aminocarbonyl” refers to a carbonyl group substituted by an amino group. Similarly, the term “alkylaminocarbonyl” refers to a carbonyl group substituted by an alkylamino group, and the term “dialkylaminocarbonyl” refers to a carbonyl group substituted by a dialkylamino group.

As used herein, the term “acyl” refers to a carbonyl group substituted by an alkyl, alkenyl, alkynyl, or carbocyclyl group.

As used herein, the term “alkylsulfonyl” refers to a sulfonyl group (SO₂) substituted by an alkyl group. Similarly, the term “arylsulfonyl” refers to a sulfonyl group substituted by an aryl group, and the term “haloalkylsulfonyl” refers to a sulfonyl group substituted by a haloalkyl group.

As used herein, the term “aminosulfonyl” refers to a sulfonyl group substituted by an amino group. Similarly, the term “alkylaminosulfonyl” refers to a sulfonyl group substituted by an alkylamino group, and the term “dialkylaminosulfonyl” refers to a sulfonyl group substituted by a dialkylamino group.

As used herein, the term “alkylsulfinyl” refers to a sulfinyl group (SO) substituted by an alkyl group. Similarly, the term “arylsulfinyl” refers to a sulfinyl group substituted by an aryl group, and the term “haloalkylsulfinyl” refers to a sulfinyl group substituted by a haloalkyl group.

As used herein, the term “ureido” refers to NHCONH₂. Similarly, the term “alkylureido” refers to a ureido group substituted by an alkyl group, and the term “dialkylureido” refers to a ureido group substituted by two alkyl groups.

As used herein, the term “thioureido” refers to NHCSNH₂. Similarly, the term “alkylthioureido” refers to a thioureido group substituted by an alkyl group, and the term “dialkylthioureido” refers to a thioureido group substituted by two alkyl groups.

As used herein, the phrase “at each independent occurrence” or the term “independently” is meant to indicate that different moieties can be selected from the recited group in the event a variable occurs more than once in a structure.

As used herein, “substituted” indicates that at least one hydrogen atom of a chemical group is replaced by a non-hydrogen moiety. When a chemical group herein is “substituted” it may have up to the full valance of substitution, provided the resulting compound is a stable compound or stable structure; for example, a methyl group may be substituted by 1, 2, or 3 substituents, a methylene group may be substituted by 1 or 2 substituents, a phenyl group may be substituted by 1, 2, 3, 4, or 5 substituents, and the like.

As used herein “stable compound” or “stable structure” refers to a compound that is sufficiently robust to survive isolation to a useful degree of purity from a reaction mixture, and preferably capable of formulation into an efficacious therapeutic agent. The present invention is directed only to stable compounds.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention can also include tautomeric forms, such as keto-enol tautomers. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium.

The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The present invention also includes prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Synthesis

Compounds of the invention, including salts, hydrates, and solvates thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Preparation of compounds of the invention can involve the protection and deprotection of various chemical groups. The need for protection and deprotection, and the selection of appropriate protecting groups can be readily determined by one skilled in the art. The chemistry of protecting groups can be found, for example, in T. W. Green and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Example synthetic routes to compounds of the invention are provided in Schemes I and II below, where constituent members of the depicted formulae are defined herein.

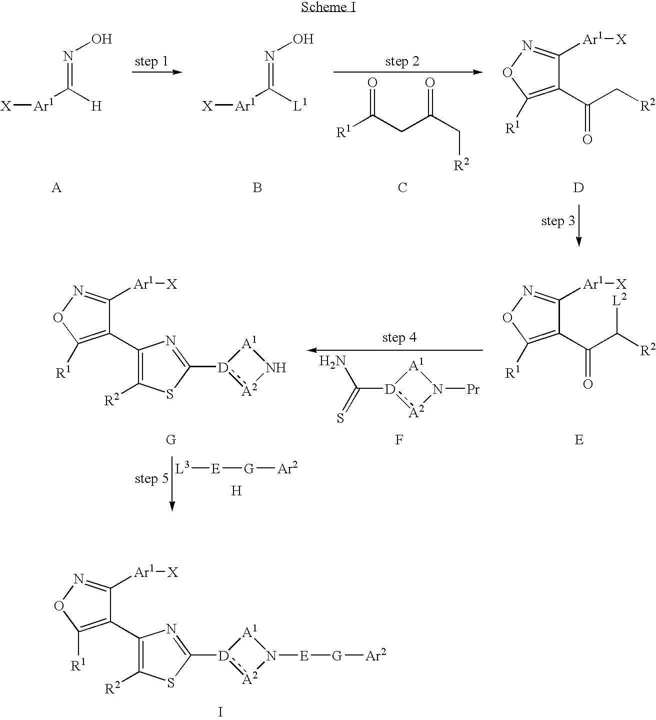

The example synthetic route of Scheme I begins with the reaction of oxime A, where X is a halogen atom, with a reagent that replaces the alpha-H of A with a suitable leaving group, L¹ to form the alpha-L₁ oxime intermediate B. In some embodiments, leaving group L¹ is a halogen atom such as F, Cl, Br or I. For example, when L¹ is Cl, oxime A can be treated with a chlorinated hydrocarbon such as CHCl₃ in the presence of an organic base and in the presence of isothiocyanate for a time and under conditions to form the alpha-chloro oxime encompassed by intermediate B. Suitable organic bases include, for example, trialkylamines (e.g., triethylamine, diisopropylethylamine, etc.), cyclic amines (e.g., pyridine, piperidine, morpholine, etc.), and the like.

Acyl-isoxazole intermediate D can be formed from the alpha-L¹ oxime B by addition of a dione (reactant C) in the presence of an organic base such as, for example, a trialkylamine (e.g., triethylamine, diisopropylethylamine, etc.), a cyclic amine (e.g., pyridine, piperidine, morpholine, etc.), combinations thereof and the like. Suitable solvents can effectively stabilize polar intermediates and include, for example, polar solvents such alcohols (e.g., methanol, ethanol, etc.).

Acyl-isoxazole intermediate D can be activated by treatment with a reagent that effectively adds a leaving group (L²) to the acyl moiety to form activated-isoxazole E. In some embodiments, leaving group L² can be a halogen atom such as F, Cl, Br or I or other leaving group such as —O-methanesulfonyl, —O-trifluoromethanesulfonyl, —O-p-toluenesulfonyl, and the like. In embodiments where L² is Br, acyl-isoxazole D can be treated with an excess amount of the Br₂ or other brominating reagent optionally in the presence of an acid, including organic acids such as acetic acid, propionic acid, trifluoroacetic acid, methylsulfonic acid, trifluoromethylsulfonic acid and the like, as well as inorganic acids including sulfuric acid, hydrochloric acid, and the like.

Formation of the thiazole moiety of intermediate G can be carried out by reaction of activated acyl-isoxazole E with an appropriate thiocarboxamide reagent (F). The ring-forming amine of the thiocarboxamide reagent (F) can be optionally protected with any suitable amine protecting group (Pr) such as BOC or other related moieties during the reaction which can be subsequently selectively removed according to well known methods in the art. Suitable amine protecting groups are described, for example, in Green et al., supra.

Intermediate I can be prepared by reaction of intermediate G with reagent L³-E-G-Ar² (H), where L³ is a leaving group and E, G, and Ar₂ are defined herein. The reaction can proceed by nucleophilic substitution, whereby the amino moiety of intermediate G replaces the leaving group L³ of the reactant (B). L³ can be any of a wide range of leaving groups including F, Cl, Br, I, OH, O-(alkyl), and the like. Suitable reaction conditions, solvents, catalysts and other parameters can be, for example, selected for compatibility with a nucleophilic substitution mechanism.

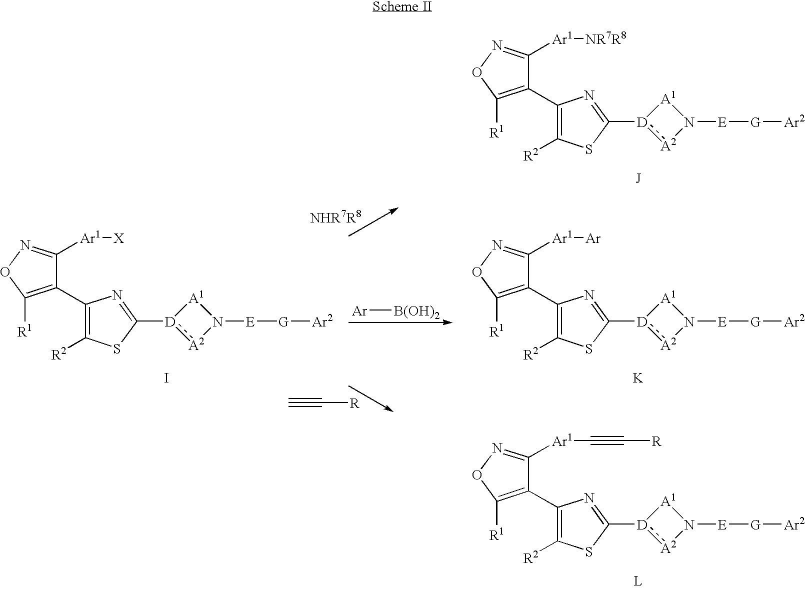

The X-substituted Ar¹ moiety of intermediate I, allows for the attachment of various substituents. For example, as depicted in Scheme II (upper), reaction of intermediate I with an aminating reagent (e.g., a primary or secondary amine such as NHR⁷R⁸) allows for the replacement of X with a wide array of amine groups according to standard amination chemistry to form compounds of the invention having the structure J. The amination reaction can be optionally catalyzed with any suitable catalyst such as a metal catalyst. Some suitable catalysts contain Pd or other heavy metals or transition metals. The amination can also be carried out in the presence of a base such as an alkoxide or other strong base.

Suzuki coupling can also lead to a variety of compounds according to the invention. For example, as depicted in Scheme II (middle), reaction of intermediate I with an aryl or heteroaryl boronic acid (Ar—B(OH)₂, where Ar is substituted or unsubstituted aryl or heteroaryl) can lead to replacement of X with a wide variety of optionally substituted aryl or heteroaryl moieties to form compounds having structure K. Typical Suzuki reaction conditions are known in the art and can include, for example, reactions at elevated temperature run in the presence of a transition metal catalyst (e.g., Pd) and/or a weak base such as sodium carbonate.

Numerous other reactions known in the art that can be used to generate a further compounds. For example, alkynyl and related substitutents can be attached to Ar¹ by reaction of intermediate I with an appropriate 1-alkyne (e.g., HC≡CR, where R can be any of a variety of substituted or unsubstituted alkyl, alkenyl, alkynyl, carbocyclyl, carbocyclylalkyl, heterocyclyl, heterocyclylalkyl, and similar moieties). The reaction can be carried out in the presence of a transition metal catalyst (e.g., Pd), a copper(I) salt (e.g., CuI), and an amine such as a secondary amine (e.g., diethylamine) to form compounds having structure L.

Methods

Compounds of the invention can modulate activity of the FSH receptor. The term “modulate” is meant to refer to an ability to increase or decrease activity of a receptor. Accordingly, compounds of the invention can be used in methods of modulating the FSH receptor by contacting the receptor with any one or more of the compounds or compositions described herein. In some embodiments, compounds of the present invention are agonists of the FSH receptor. “Agonists,” as used herein, refer to agents that can stimulate activity (i.e., activate) of a target receptor (e.g., FSH receptor). In further embodiments, the compounds of the invention can be used to modulate the FSH receptor in an individual in need of modulation of the receptor by administering a modulating amount of a compound of Formula I.

Further methods of the invention include increasing the level of adenylyl cyclase activity in a cell, cell culture or tissue expressing the FSH receptor by contacting the cell, cell culture or tissue with a compound of Formula I. Contacting a cell, cell culture or tissue expressing the FSH receptor with a compound of Formula I can also result in an increase in 5′-monophosphate (cAMP) levels. Assays for detection of adenylyl cyclase activity and for measuring increased levels of cAMP are known in the art.

Methods of the invention further include methods of inducing ovulation in a female mammal by administering to the female mammal an ovulation-inducing amount of a compound or composition described herein. By “ovulation-inducing amount” is meant an amount of administered compound or composition that result in ovulation at a time that is earlier than would typically occur in the absence of administration of the compound. Such amounts can be readily determined by a physician or clinician.

Another aspect of the present invention pertains to methods of treatment (including prophylaxis) of a FSH receptor-associated disease or disorder in an individual (e.g., patient) by administering to the individual in need of such treatment a therapeutically effective amount or dose of a compound of the present invention or a pharmaceutical composition thereof. An FSH receptor-associated disease can include any disease, disorder or condition that is directly or indirectly linked to abnormal expression or activity of the FSH receptor. An FSH receptor-associated disease can also include any disease, disorder or condition that can be prevented, ameliorated, or cured by modulating FSH receptor activity.

According to some embodiments, the FSH receptor-associated disease or disorder is selected from infertility in a female or male mammal. In some embodiments, the disorder is infertility in a woman of child-bearing age.

As used herein, the term “contacting” refers to the bringing together of indicated moieties in an in vitro system or an in vivo system. For example, “contacting” the FSH receptor with a compound of the invention includes the administration of a compound of the present invention to an individual or patient, such as a human, having a 5HT_{2C} receptor, as well as, for example, introducing a compound of the invention into a sample containing a cellular or purified preparation containing the FSH receptor.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

Pharmaceutical Formulations and Dosage Forms

When employed as pharmaceuticals, the compounds of Formula I can be administered in the form of pharmaceutical compositions. These compositions can be administered by a variety of routes including oral, rectal, transdermal, subcutaneous, intravenous, intramuscular, and intranasal, and can be prepared in a manner well known in the pharmaceutical art.

This invention also includes pharmaceutical compositions which contain, as the active ingredient, one or more of the compounds of Formula I above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgement of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

Some embodiments of the present invention include a method of producing a pharmaceutical composition for “combination-therapy” comprising admixing at least one compound according to any of the compound embodiments disclosed herein, at least one additional pharmaceutical agent, and a pharmaceutically acceptable carrier. In some embodiments, the additional pharmaceutical agent is an antiestrogen compound such as Clomiphene citrate, or the additional pharmaceutical agent is human chorionic gonadotropin (hCG) or human pituitary leutinizing hormone (LH) (see e.g., Cassidenti et al. (1992) Hum. Reprod., 7: 344-348; Breckwoldt et al. (1971) Fert. Steril., 22: 451-455; and Diedrich et al. (1988) Hum. Reprod., 3: 39-44).

Labeled Compounds and Assay Methods

Another aspect of the present invention relates to radio-labeled compounds of Formula I that would be useful not only in radio-imaging but also in assays, both in vitro and in vivo, for localizing and quantitating the FSH receptor in tissue samples, including human, and for identifying FSH receptor ligands by inhibition binding of a radio-labeled compound. Accordingly, the present invention includes FSH receptor assays that contain such radio-labeled compounds.

The present invention further includes isotopically-labeled compounds of Formula I. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro 5HT_{2C} receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound that has incorporated at least one radionuclide. In some embodiments the radionuclide is selected from the group consisting of ³H, ¹⁴C, ¹²⁵I, ³⁵S and ⁸²Br.

Synthetic methods for incorporating radio-isotopes into organic compounds are applicable to compounds of the invention and are well known in the art.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to the FSH receptor. Accordingly, the ability of a test compound to compete with the radio-labeled compound for binding to the FSH receptor directly correlates to its binding affinity.

Kits

The present invention also includes pharmaceutical kits useful, for example, in the treatment or prevention of FSH-related diseases or disorders, such as infertility, which include one or more containers containing a pharmaceutical composition comprising a therapeutically effective amount of a compound of Formula I. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### α-Chloro-3-bromobenzaldehyde oxime

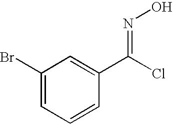

To a well stirred solution of 3-bromobenzaldehyde oxime (3.0 g, 0.015 mol) in CHCl₃ (17.0 mL) was added pyridine (0.12 mL, 1.5 mmol) followed by NCS (2.4 g, 0.018 mol). After 3 h at 40° C., the reaction mixture was cooled to room temperature and washed with water. The organic layer obtained was washed with brine, dried over MgSO₄, and concentrated in vacuo to afford the title compound (3.96 g, 100%) as a yellow solid, which was used without further purification. LCMS m/z: 234, 236 (M+H).

### Example 2

### 3-(3-Bromophenyl)-5-methyl-4-acetylisoxazole

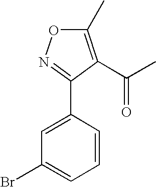

To a solution of 2,4-pentanedione (3.47 mL, 33.8 mmol) in EtOH (65.0 mL) was added Et₃N (5.0 mL, 35.9 mmol). The resulting mixture was cooled to 0° C. and added a solution of the α-chloro oxime (3.96 g, 0.017 mol) of Example 1 in EtOH (10.0 mL) dropwise via cannula. The reaction mixture was stirred at 0° C. for 1 h then at room temperature for overnight at which time the reaction mixture was quenched with H₂O and extracted with EtOAc. The combined organic layers were washed with H₂O, brine, and dried over MgSO₄ then concentrated. The crude product was purified through a silica gel column (15% EtOAc/85% hexanes) to give the title compound (3.59 g, 86% over 2 steps) as a yellow oil. LCMS m/z: 280, 282 (M+H).

### Example 3

### 3-(3-Bromophenyl)-5-methyl-4-(bromoacetyl)isoxazole

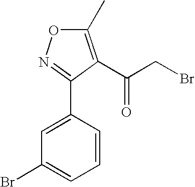

To a solution of the isoxazole of Example 2 (3.48 g, 0.012 mol) in CHCl₃ (20.0 mL) was added AcOH (0.34 mL). The resulting mixture was heated to 48° C. and Br₂ (0.50 mL, 9.76 mmol) was added in CHCl₃ (10.0 mL) via syringe. After the addition of Br₂, the reaction mixture was poured into H₂O and extracted with CH₂Cl₂. The combined organic layers were washed with H₂O, brine, dried over MgSO₄, and concentrated. The crude product was purified through a silica gel column (20% EtOAc/80% hexanes) to give a mixture (3.87 g) of the title compound and α,α-dibromo ketone (separated in the subsequent step) as a yellow oil. LCMS m/z: 358, 360, 362 (M+H).

### Example 4

### 2-Carbamoyl-pyrrolidine-1-carboxylic acid tert-butyl ester

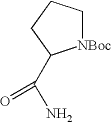

To a solution of H-DL-proline-NH₂HCl (5.00 g, 33.2 mmol) in CH₂Cl₂ (50 mL) stirred for 10 min, was added di-tert-butyl dicarbonate (7.246 g, 33.2 mmol) and Et₃N (9.26 mL, 66.4 mmol). The mixture was allowed to stir at room temperature for 16 h. The reaction was diluted with water, and the product was extracted with EtOAc. The combined organics were dried (MgSO₄), filtered, and concentrated in vacuo to afford the title compound (5.15 g, 62%) as a pale yellow solid, which was used without further purification.

### Example 5

### 2-Thiocarbamoyl-pyrrolidine-1-carboxylic acid tert-butyl ester

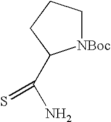

To a stirring solution of 3-carbamoyl-pyrrolidine-1-carboxylic acid tert-butyl ester (5.15 g, 24.0 mmol) of Example 4 stirred for 10 min in CH₂Cl₂ (30 mL), was added Lawesson's Reagent (10.694 g, 26.4 mmol). The mixture was allowed to stir at room temperature for 16 h. The reaction was subjected to a silica gel column using CH₂Cl₂, and eluted with EtOAc. The combined organics were concentrated in vacuo to afford the title compound (9.24 g, 100%) as a pale yellow solid, which was used without further purification. LCMS m/z: 231.2 (M+H).

### Example 6

### 3-(3-Bromo-phenyl)-5-methyl-4-(2-pyrrolidin-2-yl-thiazol-4-yl)-isoxazole Hydrobromide

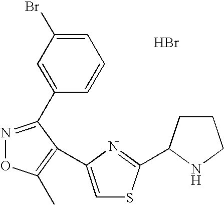

To a solution of 2-thiocarbamoyl-pyrrolidine-1-carboxylic acid-tert-butyl ester (1.53 g, 6.65 mmol) of Example 5 in ethanol (20 mL) stirring at reflux (110° C.) was added 2-bromo-1-[3-(3-bromo-phenyl)-5-methyl-isoxazol-4-yl]-ethanone (2.38 g, 6.65 mmol). The mixture was stirred at reflux for 2 h. The reaction was cooled to room temperature and the solvent was removed in vacuo. The crude product was dissolved in minimal amount of MeOH and slowly added to a stirring solution of Et₂O (50 mL). The resulting precipitate was collected to afford the title compound (1.26 g, 49%) as a white solid (HBr Salt).

LCMS m/z: 390.2, 392.2 (+H), ¹H-NMR (400 MHz; MeOD): δ 2.11-2.20 (m, 2H), 2.22-2.33 (m, 1H), 2.53-2.63 (m, 4H), 3.37-3.42 (ddd, J=7.46, 7.46, 2.61 Hz, 2H), 5.13-5.18 (t, J=7.32 Hz, 1H), 7.34-7.39 (m, 1H), 7.45-7.49 (m, 1H), 7.55 (s, 1H), 7.62-7.66 (m, 2H).

### Example 7

### 2-(4-Piperidine)-4-[3-(3-bromophenyl)-5-methylisoxazolyl]thiazole Hydrobromide

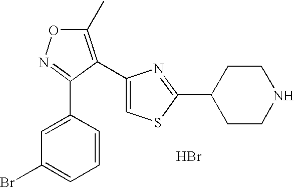

A mixture of 3-(3-bromophenyl)-5-methyl-4-(bromoacetyl)isoxazole (1.50 g, 4.18 mmol) of Example 3 and 4-thiocarbamoyl-piperidine-1-carboxylic acid-tert-butyl ester (1.3 equiv) in EtOH (14.0 mL) was heated to reflux. After 2 h, the reaction mixture was cooled to room temperature and poured into Et₂O. The product precipitated out and was filtered and dried in vacuo to give the title compound (1.48 g, 89%) as an off-white solid (HBr salt). LCMS m/z: 404, 406 (M+H).

### Example 8

### 4-{4-[3-(3-Bromo-phenyl)-5-methyl-isoxazol-4-yl]-thiazol-2-yl}-1-phenylmethane-sulfonyl-piperidine

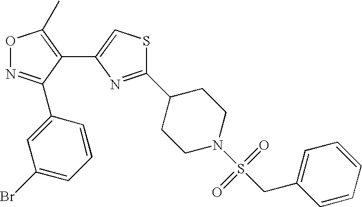

4-{4-[3-(3-Bromo-phenyl)-5-methyl-isoxazol-4-yl]-thiazol-2-yl}-piperidine hydrobromide salt from Example 7 (0.150 g, 0.461 mmol) was dissolved in 10 mL of dichloromethane. Diisopropylethylamine (0.241 mL, 1.38 mmol) was added to the solution and stirred at room temperature for 2 min. Alpha-toluenesulfonyl chloride (0.088 g, 0.461 mmol) was added and stirred for 30 min. The solvent was removed under reduced pressure and the crude material was recrystallized in methanol, yielding 0.237 g (92%) of white crystalline product.

LCMS m/z: 560.0 (M+H).

### Example 9

### 1-(4-{4-[3-(3-Bromo-phenyl)-5-methyl-isoxazol-4-yl]-thiazol-2-yl}-piperidin-1-yl)-2-thiophen-2-yl-ethanone Trifluoroacetic acid

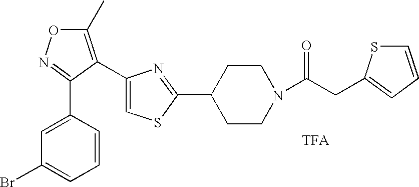

To a reaction vial was added thiophen-2-yl-acetic acid (101.7 mg, 0.252 mmol, 1.9 equiv) and PS-Carbodiimide (2.0 equiv, Argonaut) in CH₂Cl₂ (10 mL/g resin) and the mixture was stirred at room temperature. After 10 min, 3-(3-bromophenyl)-5-methyl-4-(bromoacetyl)isoxazole (1.0 equiv) of Example 7 was added to the reaction mixture. After stirring overnight at room temperature, the reaction mixture was filtered and resin was washed with CH₂Cl₂. The filtrate obtained was concentrated and the crude product was purified thought a silica gel column (EtOAc/hexanes) and further purified by HPLC (Phenomenex, Luna, 10μ C18/2 or VYDAC, Protein & Peptide C18, 0.1% TFA in acetonitrile/0.1% TFA in water) to give the title compound (56 mg, 42%) as an off white solid.

LCMS m/z: 528, 530 (M+H). ¹H-NMR (400 MHz, CD₃OD): δ 1.57 (ddd, 1H, J=3.9, 11.3, 24.7), 1.67 (ddd, 1H, J=4.1, 11.5, 24.7 Hz), 2.00-2.12 (m, 2H), 2.53 (s, 3H), 2.90 (m, 1H), 3.22-3.33 (m, 2H), 3.93 (d, 1H, J=15.7 Hz, AB system), 4.03 (d, 1H, J=15.3 Hz, AB system), 4.06 (m, 1H), 4.46 (m, 1H), 6.91-6.94 (m, 2H), 7.24-7.30 (m, 3H), 7.46 (ddd, 1H, J=1.4, 1.4, 7.8 Hz), 7.56 (ddd, 1H, J=1.2, 2.0, 7.8 Hz), 7.59 (dd, 1H, J=1.6, 1.6 Hz).

### Example 10

### 1-(4-{4-[3-(3-Bromo-phenyl)-5-methyl-isoxazol-4-yl]-thiazol-2-yl}-piperidin-1-yl)-2-thiophen-3-yl-ethanone Trifluoroacetic acid

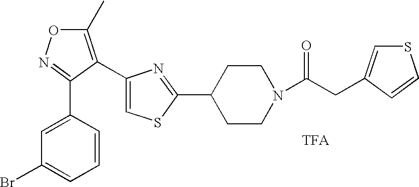

The title compound was prepared substantially as provided in Example 9 except that thiophen-3-yl-acetic acid was used to give the product (62 mg, 0.12 mmol, 47%) as a viscous oil (TFA salt).

LCMS m/z: 528, 530 (M+H). ¹H-NMR (400 MHz, CD₃OD): δ 1.49 (m, 1H), 1.66 (m, 1H), 1.98 (m, 1H), 2.08 (m, 1H), 2.53 (s, 3H), 2.89 (m, 1H), 3.18-3.28 (m, 2H), 3.74 (d, 1H, J=15.3 Hz, AB system), 3.83 (d, 1H, J=14.9 Hz, AB system), 4.01 (m, 1H), 4.47 (m, 1H), 7.00 (dd, 1H, J=1.4, 4.9 Hz), 7.16-7.17 (m, 1H), 7.27-7.31 (m, 2H), 7.35 (dd, 1H, J=2.9, 4.9 Hz), 7.46 (ddd, 1H, J=1.4, 1.4, 8.0 Hz), 7.56 (ddd, 1H, J=1.2, 2.0, 7.8 Hz), 7.59 (dd, 1H, J=1.6, 1.6 Hz).

### Example 11

### 1-(2-{4-[3-(3-Bromo-phenyl)-5-methyl-isoxazol-4-yl]-thiazol-2-yl}-pyrrolidin-1-yl)-2-thiophen-2-yl-ethanone Trifluoroacetic acid

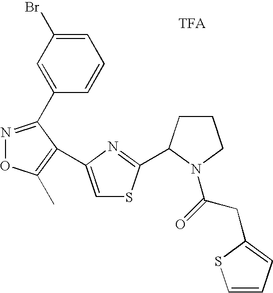

The title compound was prepared substantially as provided in Example 9 except that 3-(3-bromo-phenyl)-5-methyl-4-(2-pyrrolidin-2-yl-thiazol-4-yl)-isoxazole hydrobromide of Example 6 was used to give crude product (1.49 g, 90%) which was purified by HPLC (0.1% TFA in acetonitrile/0.1% TFA in water) to obtain the title compound as a viscous oil (TFA salt).

LCMS m/z: 514.2, 516.0 (M+H). ¹H-NMR (400 MHz; MeOD): δ 1.96-2.08 (m, 2H), 2.14-2.22 (m, 1H), 2.52 (s, 3H), 2.56 (s, 1H) 3.64-3.82 (m, 2H), 3.88-4.02 (dd, J=26.2, 16.2 Hz, 2H), 5.46-5.52 (dd, J=8.00, 1.20 Hz, 1H), 6.90-6.96 (m, 2H), 7.24-7.32 (m, 3H), 7.38-7.50 (m, 2H), 7.54-7.60 (m, 2H).

### Example 12

### 1-(4-{4-[3-(3-Isopropylamino-phenyl)-5-methyl-isoxazol-4-yl}-thiazol-2-yl]-piperidin-1-yl)-2-thiophen-2-yl-ethanone Trifluoroacetic acid

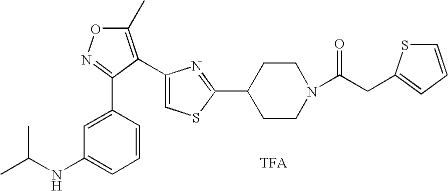

To a round bottom flask was added the 3-bromophenyl isoxazole of Example 10 (370 mg, 0.700 mmol, 1.0 equiv), isopropylamine (13 equiv), sodium tert-butoxide (1.2 equiv), and Pd[P(t-Bu)₃]₂ (10 mol %) in toluene (18.0 mL). The resulting mixture was heated at 105° C. for 17 h and at which time the reaction mixture was cooled to room temperature. The reaction mixture was washed with H₂O and extracted with EtOAc. The combined organic layers were washed with brine and dried over MgSO₄ then concentrated. The crude product obtained was purified through a silica gel column (EtOAc/hexanes) and further purified with HPLC (Phenomenex, Luna, 10μ, C18/2, 0.1% TFA in acetonitrile/0.1% TFA in water) to give the title compound (144 mg, 41%) as a white solid (TFA salt).

LCMS m/z: 507 (M+H). ¹H-NMR (400 MHz, CD₃OD): δ 1.29 (s, 3H), 1.31 (s, 3H), 1.48-1.67 (m, 2H), 2.00-2.09 (m, 2H), 2.57 (s, 3H), 2.84-2.92 (m, 1H), 3.24-3.32 (m, 2H), 3.73 (septet, 1H, J=6.7 Hz), 3.95 (d, 1H, J=16.0 Hz, AB system), 4.05 (d, 1H, J=16.0 Hz, AB system), 4.06-4.12 (m, 1H), 4.44-4.51 (m, 1H), 6.93-6.98 (m, 2H), 7.29 (dd, 1H, J=1.3, 4.0 Hz), 7.31 (s, 1H), 7.43-7.58 (m, 4H).

### Example 13

### 1-[4-(4-{5-Methyl-3-[3-(2-morpholin-4-yl-ethylamino)-phenyl]-isoxazol-4-yl}-thiazol-2-yl)-piperidin-1-yl]-2-thiophen-2-yl-ethanone Trifluoroacetic acid

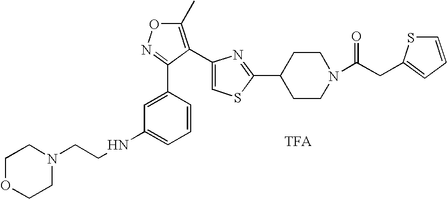

The title compound was prepared according to the Pd-catalyzed amidation procedure of Example 12 except that 2-morpholin-4-yl-ethylamine was used as the amine to obtain the title compound (50 mg, 38%) as a viscous solid (TFA salt).

LCMS m/z: 578 (M+H). ¹H-NMR (400 MHz, CD₃OD): δ 1.53-1.71 (m, 2H), 1.99-2.11 (m, 2H), 2.57 (s, 3H), 2.91-2.98 (m, 1H), 3.25-3.41 (m, 9H), 3.54 (t, 2H, J=6.2 Hz), 3.80-4.10 (m, 4H), 3.95 (d, 1H, J=15.7 Hz, AB system), 4.05 (d, 1H, J=15.5 Hz, AB system), 4.38-4.45 (m, 1H), 6.68 (d, 1H, J=7.6 Hz), 6.78 (dd, 1H, J=2.4, 8.2 Hz), 6.84 (m, 1H), 6.94-6.98 (m, 2H), 7.17 (s, 1H), 7.17 (dd, 1H, J=7.9, 7.9 Hz), 7.29 (dd, 1H, J=1.4, 4.9 Hz).

### Example 14

### 1-(4-{4-[5-Methyl-3-(3-morpholin-4-yl-phenyl)-isoxazol-4-yl]-thiazol-2-yl}-piperidin-1-yl)-2-thiophen-2-yl-ethanone Trifluoroacetic acid

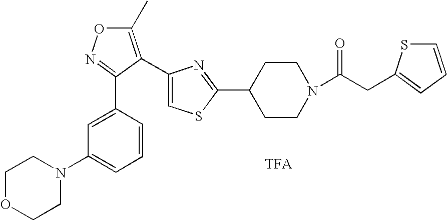

The title compound was prepared according to the Pd-catalyzed amidation procedure of Example 12 except that morpholine was used as the amine to obtain the title compound (38 mg, 33%) as a viscous solid (TFA salt).

LCMS m/z: 535 (M+H). ¹H-NMR (400 MHz, CD₃OD): δ 1.48-1.69 (m, 2H), 1.97-2.10 (m, 2H), 2.52 (s, 3H), 2.84-2.92 (m, 1H), 3.12-3.16 (m, 4H), 3.21-3.29 (m, 2H), 3.78-3.82 (m, 4H), 3.92 (d, 1H, J=15.3 Hz, AB system), 4.00-4.08 (m, 1H), 4.02 (d, 1H, J=15.7 Hz, AB system), 4.41-4.48 (m, 1H), 6.90-6.94 (m, 2H), 7.02 (ddd, 1H, J=1.2, 1.2, 7.4 Hz), 7.10-7.12 (m, 1H), 7.13-7.14 (m, 1H), 7.19 (s, 1H), 7.25 (dd, 1H, J=1.6, 4.7 Hz), 7.27-7.32 (m, 1H).

### Example 15

### 1-[4-(4-{5-Methyl-3-[3-(4-methyl-piperazin-1-yl)-phenyl]-isoxazol-4-yl}-thiazol-2-yl)-piperidin-1-yl]-2-thiophen-2-yl-ethanone Trifluoroacetic acid

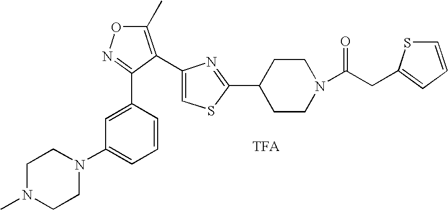

The title compound was prepared according to the Pd-catalyzed amidation procedure of Example 12 except that 1-methyl-piperazine was used as the amine to obtain the title compound (18 mg, 21%) as a white solid (TFA salt).

LCMS m/z: 548 (M+H). ¹H-NMR (400 MHz, CD₃OD): δ 1.52-1.70 (m, 2H), 1.99-2.10 (m, 2H), 2.56 (s, 3H), 2.89-3.08 (m, 3H), 2.96 (s, 3H), 3.19-3.34 (m, 4H), 3.54-3.62 (m, 2H), 3.75-3.85 (m, 2H), 3.95 (d, 1H, J=15.6 Hz, AB system), 4.02-4.09 (m, 1H), 4.05 (d, 1H, J=15.5 Hz, AB system), 4.39-4.46 (m, 1H), 6.93-6.98 (m, 3H), 7.11 (dd, 1H, J=2.5, 8.3 Hz), 7.14-7.16 (m, 1H), 7.22 (s, 1H), 7.27-7.33 (m, 2H).

### Example 16

### N-[3′-(5-Methyl-4-{2-[1-(2-thiophen-2-yl-acetyl)-piperidin-4-yl]-thiazol-4-yl}-isoxazol-3-yl)-biphenyl-4-yl]-acetamide Trifluoroacetic acid

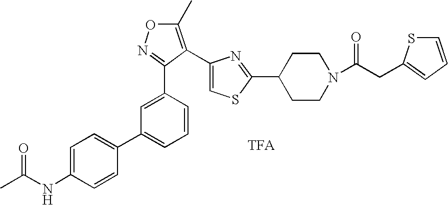

To a Smith Synthesizer vial was added 2-thiophene-3-bromophenyl isoxazole of Example 9 (72.4 mg, 0.137 mmol), 4-acetamidophenylboronic acid (51.1 mg, 0.286 mmol), aq. Na₂CO₃ (0.114 mL, 0.227 mmol, 2 M solution), and Pd(PPh₃)₄ (13.3 mg, 0.012 mmol) in a mixture of EtOH (0.18 mL) and toluene (0.69 mL). The resulting reaction mixture was heated in the Smith Synthesizer at 100° C. for 1.5 h. The reaction mixture was quenched with H₂O and extracted with EtOAc. The combined organic layers were washed with H₂O, brine, and dried over MgSO₄ then concentrated. The crude product was purified with HPLC (0.1% TFA in acetonitrile/0.1% TFA in water) to obtain the title compound (34 mg, 43%) as a TFA salt.

LCMS m/z: 583 (M+H). ¹H-NMR (400 MHz, CD₃OD): δ 1.41-1.61 (m, 2H), 1.87-2.01 (m, 2H), 2.13 (s, 3H), 2.53 (s, 3H), 2.80-2.88 (m, 1H), 3.15-3.28 (m, 2H), 3.91 (d, 1H, J=15.7 Hz, AB system), 3.92-4.01 (m, 1H), 3.99 (d, 1H, J=15.6 Hz, AB system), 4.35-4.42 (m, 1H), 6.90-6.95 (m, 2H), 7.26 (dd, 1H, J=1.2, 5.1 Hz), 7.33 (s, 1H), 7.40-7.53 (m, 4H), 7.58-7.68 (m, 4H).

### Example 17

### N-[3′-(5-Methyl-4-{2-[1-(2-thiophen-2-yl-acetyl)-piperidin-4-yl]-thiazol-4-yl}-isoxazol-3-yl)-biphenyl-3-yl]-acetamide Trifluoroacetic acid

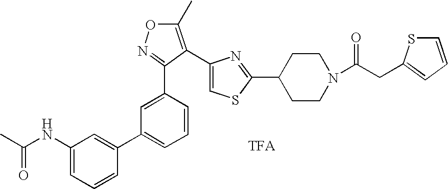

This compound was prepared according to the procedure of Example 16 except that 3-acetamidophenylboronic acid was used to obtain the title compound (36 mg, 54%) as a white solid (TFA salt).

LCMS m/z: 583 (M+H). ¹H-NMR (400 MHz, CD₃OD): δ 1.39-1.58 (m, 2H), 1.82-1.98 (m, 2H), 2.13 (s, 3H), 2.54 (s, 3H), 2.78-2.86 (m, 1H), 3.13-3.25 (m, 2H), 3.89 (d, 1H, J=16.0 Hz, AB system), 3.90-3.96 (m, 1H), 3.96 (d, 1H, J=15.7 Hz, AB system), 4.31-4.38 (m, 1H), 6.89 (dd, 1H, J=1.2, 3.5 Hz), 6.91 (dd, 1H, J=3.5, 5.1 Hz), 7.16-7.19 (m, 1H), 7.24 (dd, 1H, J=1.2, 5.1 Hz), 7.30 (s, 1H), 7.32 (dd, 1H, J=8.0, 8.0 Hz), 7.43-7.51 (m, 3H), 7.60 (dd, 1H, J=1.4, 1.4 Hz), 7.64 (ddd, 1H, J=1.8, 1.8, 7.6 Hz), 7.75 (dd, 1H, J=2.0, 2.0 Hz).

### Example 18

### 3′-(5-Methyl-4-{2-[1-(2-thiophen-2-yl-acetyl)-piperidin-4-yl]-thiazol-4-yl}-isoxazol-3-yl)-biphenyl-4-carboxylic acid amide Trifluoroacetic acid

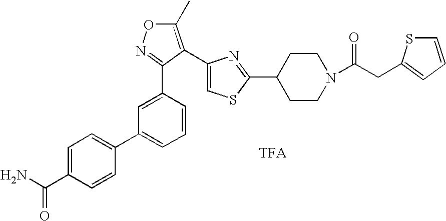

This compound was prepared according to the procedure of Example 16 except that 4-benzamide boronic acid was used to obtain the title compound (43 mg, 51%) as a white solid (TFA salt).

LCMS m/z: 569 (M+H). ¹H-NMR (400 MHz, CD₃OD): δ 1.40-1.58 (m, 2H), 1.86-1.98 (m, 2H), 2.54 (s, 3H), 2.78-2.86 (m, 1H), 3.15-3.27 (m, 2H), 3.90 (d, 1H, J=15.3 Hz, AB system), 3.93-3.99 (m, 1H), 3.99 (d, 1H, J=15.7 Hz, AB system), 4.32-4.39 (m, 1H), 6.88-6.93 (m, 2H), 7.23 (dd, 1H, J=1.4, 4.9 Hz), 7.33 (s, 1H), 7.49 (dd, 1H, J=7.6, 7.6 Hz), 7.53-7.58 (m, 3H), 7.64-7.65 (m, 1H), 7.72 (ddd, 1H, J=1.6, 1.6, 7.7 Hz), 7.89 (dd, 1H, J=2.0, 2.0 Hz), 7.92 (dd, 1H, J=2.0, 2.0 Hz).

### Example 19

### 3′-(5-Methyl-4-{2-[1-(2-thiophen-2-yl-acetyl)-piperidin-4-yl]-thiazol-4-yl}-isoxazol-3-yl)-biphenyl-3-carboxylic acid amide Trifluoroacetic acid

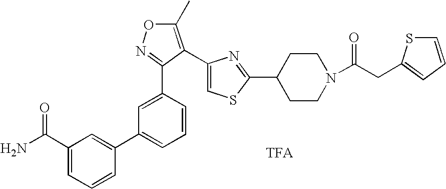

This compound was prepared according to the procedure of Example 16 except that 3-benzamide boronic acid was used to obtain the title compound (22 mg, 34%) as a white solid (TFA salt).

LCMS m/z: 569 (+H). ¹H-NMR (400 MHz, CD₃OD): δ 1.41-1.59 (m, 2H), 1.89-1.99 (m, 2H), 2.54 (s, 3H), 2.79-2.86 (m, 1H), 3.15-3.27 (m, 2H), 3.90 (d, 1H, J=15.7 Hz, AB system), 3.93-3.99 (m, 1H), 3.98 (d, 1H, J=15.7 Hz, AB system), 4.33-4.40 (m, 1H), 6.88-6.93 (m, 2H), 7.24 (dd, 1H, J=1.2, 5.1 Hz), 7.31 (s, 1H), 7.47-7.52 (m, 2H), 7.54 (ddd, 1H, J=1.4, 1.4, 7.6 Hz), 7.65 (dd, 1H, J=1.4, 1.4 Hz), 7.67-7.68 (m, 1H), 7.72 (ddd, 1H, J=1.4, 1.4, 7.4 Hz), 7.82-7.85 (m, 1H), 8.00 (dd, 1H, J=1.6, 1.6 Hz).

### Example 20

### 3′-{5-Methyl-4-[2-(1-phenylmethanesulfonyl-piperidin-4-yl)-thiazol-4-yl]-isoxazol-3-yl}-biphenyl-4-carboxylic acid Trifluoroacetic acid

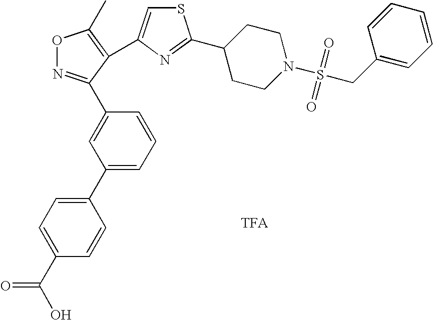

This compound was prepared according to the procedure of Example 16 except that 4-{4-[3-(3-bromo-phenyl)-5-methyl-isoxazol-4-yl]-thiazol-2-yl}-1-phenylmethane-sulfonyl-piperidine of Example 8 and 4-carboxyphenyl boronic acid was used to obtain the title compound (0.126 g, 42%) as a white solid (TFA salt).

LCMS m/z 600.4 (M+H); ¹H-NMR (400 MHz, DMSO) δ 8.02 (d, J=8.0 Hz, 2H), 7.86-7.83 (m, 1H), 7.82-7.81 (m, 1H), 7.71 (d, J=4.0 Hz, 2H), 7.61-7.58 (m, 2H), 7.53 (s, 1H), 7.41-7.34 (m, 5H), 4.37 (s, 2H), 3.50 (d, J=12.0 Hz, 2H), 3.10 (tt, J=12.0, 4.0 Hz, 1H), 2.82 (td, J=12.0, 4.0 Hz, 2H), 2.58 (s, 3H), 1.92 (dd, J=16.0, 4.0 Hz, 2H), 1.55-1.45 (m, 2H).

### Example 21

### 3′-(5-Methyl-4-{2-[1-(2-thiophen-2-yl-acetyl)-piperidin-4-yl]-thiazol-4-yl}-isoxazol-3-yl)-biphenyl-4-carboxylic acid Trifluoroacetic acid

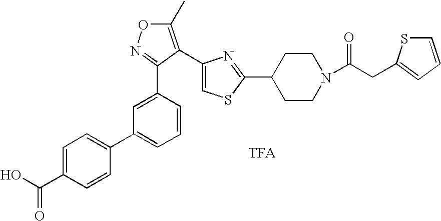

This compound was prepared according to the procedure of Example 16 except that 4-carboxyphenyl boronic acid was used to obtain the title compound (27 mg, 39%) as a white solid (TFA salt).

LCMS m/z: 570 (M+H). ¹H-NMR (400 MHz, CD₃OD): δ 1.40-1.63 (m, 2H), 1.86-2.02 (m, 2H), 2.55 (s, 3H), 2.80-2.89 (m, 1H), 3.16-3.27 (m, 2H), 3.91 (d, 1H, J=15.6 Hz, AB system), 3.93-4.00 (m, 1H), 4.00 (d, 1H, J=15.5 Hz, AB system), 4.36-4.43 (m, 1H), 6.91-6.95 (m, 2H), 7.26 (dd, 1H, J=1.3, 5.0 Hz), 7.35 (s, 1H), 7.50-7.63 (m, 4H), 7.68-7.69 (m, 1H), 7.76 (ddd, 1H, J=1.5, 1.5, 7.6 Hz), 8.06-8.10 (m, 2H).

### Example 22

### 3′-(5-Methyl-4-{2-[1-(2-thiophen-2-yl-acetyl)-pyrrolidin-2-yl]-thiazol-4-yl}-isoxazol-3-yl)-biphenyl-4-carboxylic acid Trifluoroacetic acid

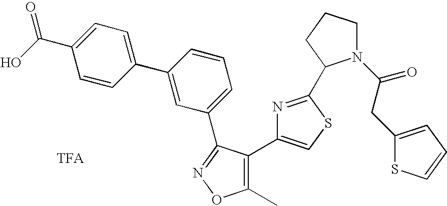

This compound was prepared according to the procedure of Example 16 except that 1-(2-{4-[3-(3-Bromo-phenyl)-5-methyl-isoxazol-4-yl]-thiazol-2-yl}-pyrrolidin-1-yl)-2-thiophen-2-yl-ethanone trifluoroacetic acid of Example 11 and 4-carboxyphenyl boronic acid was used to obtain the title compound (15 mg, 10%) as a white solid (TFA salt). LCMS m/z/z: 556 (4+H).

### Example 23

### 3′-(5-Methyl-4{-2-[1-(2-thiophen-2-yl-acetyl)-piperidin-4-yl]-thiazol-4-yl}-isoxazol-3-yl)-biphenyl-3-carboxylic acid Trifluoroacetic acid

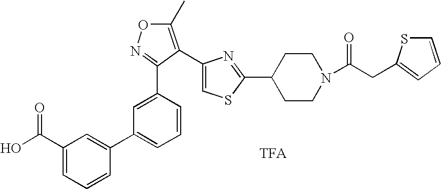

This compound was prepared according to the procedure of Example 16 except that 3-carboxyphenyl boronic acid was used to obtain the title compound (25 mg, 35%) as a white solid (TFA salt).

LCMS m/z: 570 (M+H). ¹H-NMR (400 MHz, CD₃OD): δ 1.41-1.62 (m, 2H), 1.90-2.01 (m, 2H), 2.54 (s, 3H), 2.79-2.86 (m, 1H), 3.12-3.35 (m, 2H), 3.90 (d, 1H, J=15.7 Hz, AB system), 3.94-4.00 (m, 1H), 3.98 (d, 1H, J=15.7 Hz, AB system), 4.36-4.43 (m, 1H), 6.88-6.94 (m, 2H), 7.23 (dd, 1H, J=1.6, 5.1 Hz), 7.35 (s, 1H), 7.48-7.63 (m, 4H), 7.71 (ddd, 1H, J=1.6, 1.6, 7.7 Hz), 7.73 (ddd, 1H, J=1.5, 1.5, 7.7 Hz), 7.99 (ddd, 1H, J=1.4, 1.4, 7.8 Hz), 8.07 (dd, 1H, J=1.8, 1.8 Hz).

### Example 24

### 1-(4-{4-[5-Methyl-3-(3-pyridin-4-yl-phenyl)-isoxazol-4-yl]-thiazol-2-yl}-piperidin-1-yl)-2-thiophen-2-yl-ethanone Trifluoroacetic acid

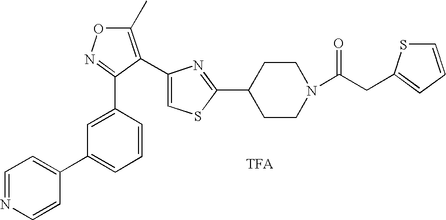

This compound was prepared according to the procedure of Example 16 except that 4-pyridyl boronic acid was used to obtain the title compound (31 mg, 37%) as a white solid (TFA salt).

LCMS m/z: 527 (M+H). ¹H-NMR (400 MHz, CD₃OD): δ 1.45-1.56 (m, 2H), 1.92-2.00 (m, 2H), 2.58 (s, 3H), 2.80-2.88 (m, 1H), 3.20-3.28 (m, 2H), 3.91 (d, 1H, J=15.7 Hz, AB system), 3.97-4.04 (m, 1H), 4.01 (d, 1H, J=15.7 Hz, AB system), 4.29-4.36 (m, 1H), 6.89-6.94 (m, 2H), 7.25 (dd, 1H, J=1.6, 5.1 Hz), 7.33 (s, 1H), 7.63 (dd, 1H, J=7.8, 15.3 Hz), 7.67 (ddd, 1H, J=1.6, 1.6, 7.8 Hz), 8.00 (ddd, 1H, J=1.6, 1.6, 7.4 Hz), 8.08 (dd, 1H, J=1.6, 1.6 Hz), 8.27 (dd, 2H, J=1.4, 5.7 Hz), 8.83 (d, 2H, J=7.0 Hz).

### Example 25

### 1-(4-{4-[5-Methyl-3-(3-pyrimidin-5-yl-phenyl)-isoxazol-4-yl]-thiazol-2-yl}-piperidin-1-yl)-2-thiophen-2-yl-ethanone Trifluoroacetic acid

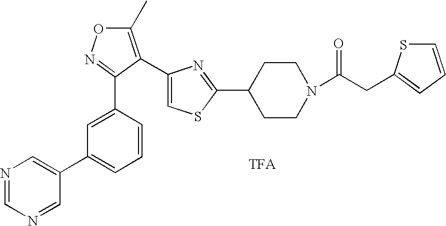

This compound was prepared according to the procedure of Example 16 except that 5-pyrimidinyl boronic acid was used to obtain the title compound (20 mg, 29%) as a white solid (TFA salt).

LCMS m/z: 528 (M+H). ¹H-NMR (400 MHz, CD₃OD): δ 1.45-1.64 (m, 2H), 1.94-2.06 (m, 2H), 2.56 (s, 3H), 2.82-2.89 (m, 1H), 3.20-3.29 (m, 2H), 3.94 (d, 1H, J=15.6 Hz, AB system), 4.01-4.07 (m, 1H), 4.03 (d, 1H, J=15.6 Hz, AB system), 4.40-4.47 (m, 1H), 6.92-6.95 (m, 2H), 7.27 (dd, 1H, J=1.7, 4.7 Hz), 7.34 (s, 1H), 7.56-7.60 (m, 1H), 7.63 (ddd, 1H, J=1.5, 1.5, 7.8 Hz), 7.75-7.82 (m, 2H), 8.98 (s, 2H), 9.16 (s, 1H).

### Example 26

### 1-(4-{4-[3-(4′-Methoxy-biphenyl-3-yl)-5-methyl-isoxazol-4-yl]-thiazol-2-yl}-piperidin-1-yl)-2-thiophen-2-yl-ethanone Trifluoroacetic acid

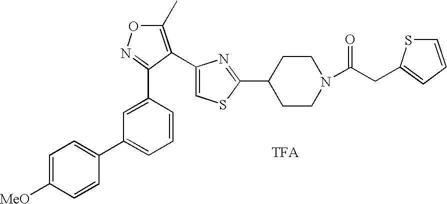

This compound was prepared according to the procedure of Example 16 except that 4-methoxyphenyl boronic acid was used to obtain the title compound (31 mg, 46%) as a white solid (TFA salt).

LCMS m/z: 556 (M+H). ¹H-NMR (400 MHz, CD₃OD): δ 1.38-1.57 (m, 2H), 1.83-1.97 (m, 2H), 2.50 (s, 3H), 2.76-2.84 (m, 1H), 3.10-3.23 (m, 2H), 3.77 (s, 3H), 3.87 (d, 1H, J=15.7 Hz, AB system), 3.88-3.95 (m, 1H), 3.95 (d, 1H, J=15.6 Hz, AB system), 4.31-4.37 (m, 1H), 6.86-6.94 (m, 4H), 7.22 (dd, 1H, J=1.1, 5.1 Hz), 7.26 (s, 1H), 7.34-7.44 (m, 4H), 7.52 (br s, 1H), 7.57-7.60 (m, 1H).

### Example 27

### 3′-(5-Methyl-4{-2-[1-(2-thiophen-2-yl-acetyl)-piperidin-4-yl]-thiazol-4-yl}-isoxazol-3-yl)-biphenyl-4-carboxylic acid dimethylamide Trifluoroacetic acid

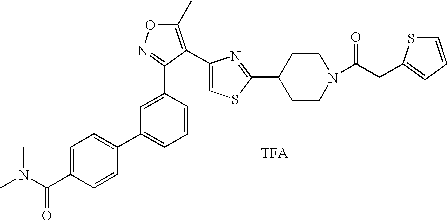

This compound was prepared according to the procedure of Example 16 except that the 4-N,N-dimethylcarbamoylphenyl boronic acid was used to obtain the title compound (57 mg, 79%) as a white solid (TFA salt).

LCMS m/z: 597 (M+H). ¹H-NMR (400 MHz, CD₃OD): δ 1.43-1.63 (m, 2H), 1.88-2.03 (m, 2H), 2.55 (s, 3H), 2.81-2.89 (m, 1H), 3.03 (s, 3H), 3.12 (s, 3H), 3.17-3.29 (m, 2H), 3.92 (d, 1H, J=15.6 Hz, AB system), 3.95-4.02 (m, 1H), 4.01 (d, 1H, J=15.6 Hz, AB system), 4.35-4.42 (m, 1H), 6.91-6.96 (m, 2H), 7.26 (dd, 1H, J=1.3, 5.0 Hz), 7.33 (s, 1H), 7.47-7.61 (m, 6H), 7.66 (m, 1H), 7.73 (ddd, 1H, J=1.5, 1.5, 7.5 Hz).

### Example 28

### 3′-(5-Methyl-4{-2-[1-(2-thiophen-2-yl-acetyl)-piperidin-4-yl]-thiazol-4-yl}-isoxazol-3-yl)-biphenyl-4-carboxylic acid methylamide Trifluoroacetic acid

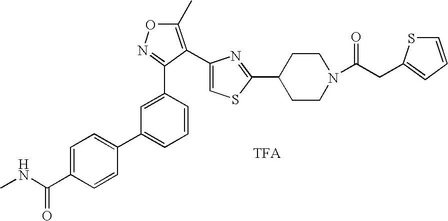

This compound was prepared according to the procedure of Example 16 except that the 4-N-methylcarbamoylphenyl boronic acid was used to obtain the title compound (30 mg, 43%) as a white solid (TFA salt).

LCMS m/z: 583 (M+H). ¹H-NMR (400 MHz, CD₃OD): δ 1.40-1.58 (m, 2H), 1.86-2.00 (m, 2H), 2.55 (s, 3H), 2.79-2.87 (m, 1H), 2.93 (s, 3H), 3.16-3.28 (m, 2H), 3.91 (d, 1H, J=15.7 Hz, AB system), 3.93-4.00 (m, 1H), 3.99 (d, 1H, J=15.5 Hz, AB system), 4.33-4.39 (m, 1H), 6.90-6.95 (m, 2H), 7.26 (dd, 1H, J=1.3, 5.1 Hz), 7.35 (s, 1H), 7.51 (dd, 1H, J=7.7, 7.7 Hz), 7.56-7.60 (m, 3H), 7.66 (dd, 1H, J=1.4, 1.4 Hz), 7.73 (ddd, 1H, J=1.5, 1.5, 7.7 Hz), 7.84-7.88 (m, 2H).

### Example 29

### 1-[4-(4-{5-Methyl-3-[4′-(morpholine-4-carbonyl)-biphenyl-3-yl]-isoxazol-4-yl}-thiazol-2-yl)-piperidin-1-yl]-2-thiophen-2-yl-ethanone Trifluoroacetic acid

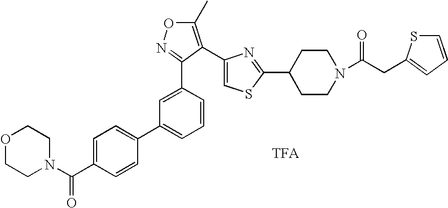

This compound was prepared according to the procedure of Example 16 except that the 4-(morpholine-4-carbonyl)phenyl boronic acid was used to obtain the title compound (48 mg, 64%) as a white solid (TFA salt).

LCMS m/z: 639 (M+H). ¹H-NMR (400 MHz, CD₃OD): δ 1.42-1.61 (m, 2H), 1.87-2.01 (m, 2H), 2.55 (s, 3H), 2.80-2.88 (m, 1H), 3.16-3.28 (m, 2H), 3.43-3.80 (m, 8H), 3.91 (d, 1H, J=15.7 Hz, AB system), 3.93-4.00 (m, 1H), 4.00 (d, 1H, J=15.7 Hz, AB system), 4.32-4.40 (m, 1H), 6.89-6.93 (m, 2H), 7.24 (dd, 1H, J=1.2, 5.1 Hz), 7.32 (s, 1H), 7.46-7.54 (m, 4H), 7.56-7.60 (m, 2H), 7.64 (dd, 1H, J=1.6, 1.6 Hz), 7.70 (ddd, 1H, J=1.8, 1.8, 7.4 Hz).

### Example 30

### 1-[4-(4-{5-Methyl-3-[3′-(morpholine-4-carbonyl)-biphenyl-3-yl]-isoxazol-4-yl}-thiazol-2-yl)-piperidin-1-yl]-2-thiophen-2-yl-ethanone Trifluoroacetic acid

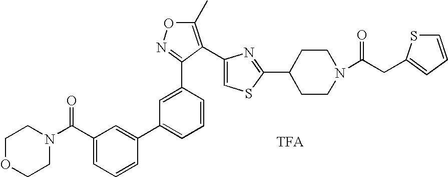

This compound was prepared according to the procedure of Example 16 except that the 3-(morpholine-4-carbonyl)phenyl boronic acid was used to obtain the title compound (41 mg, 52%) as a white solid (TFA salt).

LCMS m/z: 639 (M+H). ¹H-NMR (400 MHz, CD₃OD): δ 1.42-1.61 (m, 2H), 1.90-2.02 (m, 2H), 2.54 (s, 3H), 2.80-2.88 (m, 1H), 3.16-3.28 (m, 2H), 3.40-3.80 (m, 8H), 3.92 (d, 1H, J=15.7 Hz, AB system), 3.96-4.03 (m, 1H), 4.00 (d, 1H, J=15.3 Hz, AB system), 4.36-4.43 (m, 1H), 6.89-6.93 (m, 2H), 7.24 (dd, 1H, J=1.6, 5.1 Hz), 7.33 (s, 1H), 7.39 (ddd, 1H, J=1.4, 1.4, 7.6 Hz), 7.47-7.53 (m, 3H), 7.54 (ddd, 1H, J=1.4, 1.4, 7.8 Hz), 7.59-7.63 (m, 2H), 7.70 (ddd, 1H, J=1.7, 1.7, 7.7 Hz).

### Example 31

### 1-(4-{4-[3-(4′-Amino-biphenyl-3-yl)-5-methyl-isoxazol-4-yl]-thiazol-2-yl}-piperidin-1-yl)-2-thiophen-2-yl-ethanone Trifluoroacetic acid

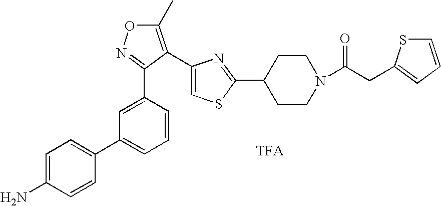

This compound was prepared according to the procedure of Example 16 except that 4-aminophenyl boronic acid was used to obtain the title compound (16 mg, 25%) as a white solid (TFA salt).

LCMS m/z: 541 (M+H). ¹H-NMR (400 MHz, CD₃OD): δ 1.37-1.57 (m, 2H), 1.88-2.00 (m, 2H), 2.55 (s, 3H), 2.78-2.86 (m, 1H), 3.19-3.27 (m, 2H), 3.92 (d, 1H, J=15.3 Hz, AB system), 3.98-4.05 (m, 1H), 4.02 (d, 1H, J=15.7 Hz, AB system), 4.25-4.32 (m, 1H), 6.90-6.94 (m, 2H), 7.26 (dd, 1H, J=1.2, 5.1 Hz), 7.33 (s, 1H), 7.38-7.42 (m, 2H), 7.47-7.53 (m, 2H), 7.64-7.71 (m, 4H).

### Example 32

### N-[3′-(5-Methyl-4-{2-[1-(2-thiophen-2-yl-acetyl)-piperidin-4-yl]-thiazol-4-yl}-isoxazol-3-yl)-biphenyl-4-yl]-methanesulfonamide Trifluoroacetic acid

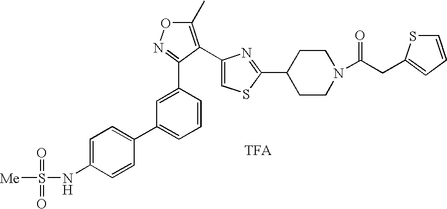

This compound was prepared according to the procedure of Example 16 except that the 4-methanesulfonylaminophenyl boronic acid was used to obtain the title compound (15 mg, 20%) as a white solid (TFA salt).

LCMS m/z: 619 (M+H). ¹H-NMR (400 MHz, CD₃OD): δ 1.43-1.61 (m, 2H), 1.90-2.01 (m, 2H), 2.54 (s, 3H), 2.81-2.89 (m, 1H), 2.98 (s, 3H), 3.12-3.35 (m, 2H), 3.91 (d, 1H, J=15.7 Hz, AB system), 3.94-4.02 (m, 1H), 4.00 (d, 1H, J=15.3 Hz, AB system), 4.34-4.40 (m, 1H), 6.89-6.93 (m, 2H), 7.24 (dd, 1H, J=1.6, 5.1 Hz), 7.26-7.30 (m, 2H), 7.32 (s, 1H), 7.43-7.47 (m, 3H), 7.49 (ddd, 1H, J=1.5, 1.5, 7.7 Hz), 7.57-7.59 (m, 1H), 7.65 (ddd, 1H, J=1.8, 1.8, 7.4 Hz).

### Example 33

### 1-(4-{4-[3-(4′-Methanesulfonyl-biphenyl-3-yl)-5-methyl-isoxazol-4-yl]-thiazol-2-yl}-piperidin-1-yl)-2-thiophen-2-yl-ethanone Trifluoroacetic acid

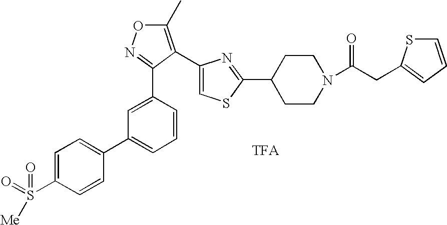

This compound was prepared according to the procedure of Example 16 except that 4-(methanesulfonyl)-phenyl boronic acid was used to obtain the title compound (12 mg, 17%) as a white solid (TFA salt).

LCMS m/z: 604 (M+H). ¹H-NMR (400 MHz, CD₃OD): δ 1.41-1.60 (m, 2H), 1.87-2.01 (m, 2H), 2.55 (s, 3H), 2.80-2.88 (m, 1H), 3.15 (s, 3H), 3.16-3.32 (m, 2H), 3.91 (d, 1H, J=15.7 Hz, AB system), 3.93-4.00 (m, 1H), 4.00 (d, 1H, J=15.3 Hz, AB system), 4.31-4.39 (m, 1H), 6.89-6.94 (m, 2H), 7.24 (dd, 1H, J=1.4, 4.9 Hz), 7.33 (s, 1H), 7.52 (dd, 1H, J=7.6, 7.6 Hz), 7.58 (ddd, 1H, J=1.4, 1.4, 7.8 Hz), 7.70 (dd, 1H, J=1.6, 1.6 Hz), 7.73-7.77 (m, 3H), 7.97-8.01 (m, 2H).

### Example 34

### 2,2,2-Trifluoro-N-[3′-(5-methyl-4-{2-[1-(2-thiophen-2-yl-acetyl)-piperidin-4-yl]-thiazol-4-yl}-isoxazol-3-yl)-biphenyl-3-yl]-acetamide Trifluoroacetic acid

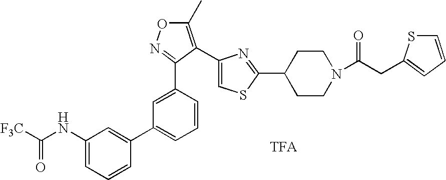

This compound was prepared according to the procedure of Example 16 except that 3-(2,2, 2-trifluoro-acetylamino)phenyl boronic acid was used to obtain the title compound (25 mg, 31%) as a white solid (TFA salt).

LCMS m/z: 637 (M+H). ¹H-NMR (400 MHz, CD₃OD): δ 1.41-1.59 (m, 2H), 1.87-2.00 (m, 2H), 2.55 (s, 3H), 2.79-2.87 (m, 1H), 3.15-3.27 (m, 2H), 3.90 (d, 1H, J=15.3 Hz, AB system), 3.93-3.99 (m, 1H), 3.98 (d, 1H, J=15.3 Hz, AB system), 4.32-4.39 (m, 1H), 6.88-6.93 (m, 2H), 7.24 (dd, 1H, J=1.2, 5.1 Hz), 7.31 (s, 1H), 7.34 (ddd, 1H, J=1.4, 1.4, 7.8 Hz), 7.42 (dd, 1H, J=7.8, 7.8 Hz), 7.48 (dd, 1H, J=7.4, 7.4 Hz), 7.53 (ddd, 1H, J=1.5, 1.5, 7.7 Hz), 7.59 (ddd, 1H, J=0.9, 2.1, 8.1 Hz), 7.61 (dd, 1H, J=1.6, 1.6 Hz), 7.67 (ddd, 1H, J=1.8, 1.8, 7.4 Hz), 7.81 (dd, 1H, J=1.8, 1.8 Hz).

### Example 35

### 1-(4-{4-[3-(3′-Methanesulfonyl-biphenyl-3-yl)-5-methyl-isoxazol-4-yl]-thiazol-2-yl}-piperidin-1-yl)-2-thiophen-2-yl-ethanone Trifluoroacetic acid

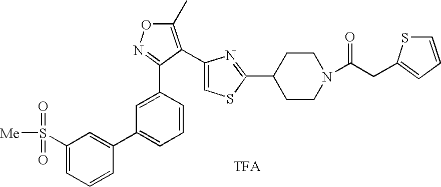

This compound was prepared according to the procedure of Example 16 except that 3-(methanesulfonyl)-phenyl boronic acid was used to obtain the title compound (15 mg, 22%) as a white solid (TFA salt).

LCMS m/z: 604 (M+H). ¹H-NMR (400 MHz, CD₃OD): δ 1.42-1.60 (m, 2H), 1.89-2.02 (m, 2H), 2.54 (s, 3H), 2.78-2.86 (m, 1H), 3.15 (s, 3H), 3.16-3.32 (m, 2H), 3.91 (d, 1H, J=15.7 Hz, AB system), 3.95-4.02 (m, 1H), 3.99 (d, 1H, J=15.7 Hz, AB system), 4.34-4.41 (m, 1H), 6.89-6.93 (m, 2H), 7.24 (dd, 1H, J=1.6, 5.1 Hz), 7.33 (s, 1H), 7.53 (dd, 1H, J=7.6, 7.6 Hz), 7.60 (ddd, 1H, J=1.4, 1.4, 7.6 Hz), 7.65 (dd, 1H, J=1.6, 1.6 Hz), 7.67 (dd, 1H, J=7.6, 7.6 Hz), 7.74 (ddd, 1H, J=1.6, 1.6, 7.6 Hz), 7.85 (ddd, 1H, J=1.0, 1.8, 7.8 Hz), 7.92 (ddd, 1H, J=1.0, 1.8, 7.8 Hz), 7.99 (dd, 1H, J=1.8, 1.8 Hz).

### Example 36

### 3′-(5-Methyl-4-{2-[1-(2-thiophen-2-yl-acetyl)-piperidin-4-yl]-thiazol-4-yl}-isoxazol-3-yl)-biphenyl-4-carbonitrile Trifluoroacetic acid

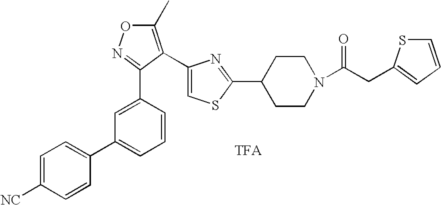

This compound was prepared according to the procedure of Example 16 except that 4-cyanophenyl boronic acid was used to obtain the title compound (16 mg, 24%) as a white solid of a TFA salt.

LCMS m/z: 551 (M+H). ¹H-NMR (400 MHz, CD₃OD): δ 1.42-1.61 (m, 2H), 1.89-2.01 (m, 2H), 2.56 (s, 3H), 2.81-2.90 (m, 1H), 3.18-3.30 (m, 2H), 3.92 (d, 1H, J=15.6 Hz, AB system), 3.96-4.03 (m, 1H), 4.02 (d, 1H, J=15.5 Hz, AB system), 4.34-4.41 (m, 1H), 6.91-6.96 (m, 2H), 7.27 (dd, 1H, J=1.3, 5.0 Hz), 7.33 (s, 1H), 7.54 (dd, 1H, J=7.4, 14.9 Hz), 7.57 (ddd, 1H, J=1.5, 1.5, 7.8 Hz), 7.68-7.81 (m, 6H).

### Example 37

### 1-{4-[4-(3-{3-[3-(1,1-Dioxo-1λ⁶-thiomorpholin-4-yl)-prop-1-ynyl]-phenyl}-5-methyl-isoxazol-4-yl)-thiazol-2-yl]-piperidin-1-yl}-2-thiophen-2-yl-ethanone

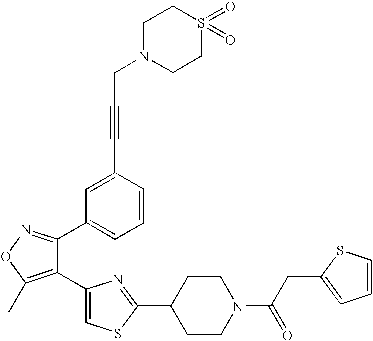

In a 5 mL Pyrex tube were placed the aryl bromide of Example 9 (100 mg, 0.189 mmol), 4-prop-2-ynyl-1λ⁶,4-thiazine-1,1-dione (173.23 mg, 0.208 mmol), diacetatobis(triphenylphosphine) palladium (3 mg, 0.004 mmol), copper iodide (1.4 mg, 0.008 mmol), diethylamine (295 μL, 2.838 mmol), 2.5 mL DMF and a magnetic stir bar. The vessel was sealed w/ a septum and irradiated at 120° C. for 15 min. After cooling to room temperature, the reaction mixture was treated w/ diethyl ether and filtered. The filtrate was poured into 0.1 M HCl (10 mL) and the resulting mixture was extracted with diethyl ether (3×10 mL). The organic layers were combined and washed with saturated sodium carbonate, extracted with diethyl ether, washed with water, then extracted with diethyl ether two times. The organic layer was condensed in vacuo. The resulting colorless oil was dissolved in ethyl acetate and filtered through celite. The filtrate was condensed in vacuo to yield crude product. The product was purified by flash chromatography (silica gel, 10 CV @ 20%, 10 CV @ 20-50%, 10 CV @ 50% acetone in hexane) to give a white solid (42 mg, 20%).

LCMS m/z 621: (+H). ¹H-NMR (400 MHz, CD₃OD) δ 7.47-7.54 (m, 5H), 7.38-7.43 (m, 1H), 7.33 (s, 1H), 6.95-6.99 (m, 2H), 4.45 (d, J=13.42 Hz, 1H), 4.05 (d, J=13.53 Hz, 1H), 3.96 (d, J=15.53 Hz, 1H), 3.80 (s, 4H), 3.32 (s, 2H), 3.22 (s, 4H) 2.92-3.00 (m, 1H), 2.56 (s, 3H), 2.00-2.13 (m, 2H), 1.59-1.73 (m, 2H).

### Example 38

### 1-[4-(4-{5-Methyl-3-[4′-(1H-tetrazol-5-yl)-biphenyl-3-yl]-isoxazol-4-yl}-thiazol-2-yl)-piperidin-1-yl]-2-thiophen-2-yl-ethanone

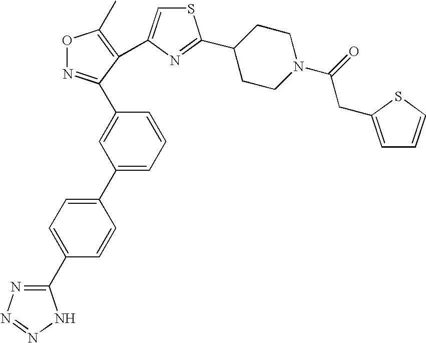

To a dry Smith synthesizer vial was added NaN₃ (0.044 g, 0.674 mmol), triethylamine hydrochloride (0.055 g, 0.674 mmol) and the mixture was sealed under argon atmosphere. Then 3′-(5-Methyl-4-{2-[1-(2-thiophen-2-yl-acetyl)-piperidin-4-yl]-thiazol-4-yl}-isoxazol-3-yl)-biphenyl-4-carbonitrile (0.040 g, 0.084 mmol, see Example 35) was dissolved in dry DMF, added to the vial, and the resulting mixture was stirred at 110° C. for 16 h. The reaction mixture was cooled to ambient temperature, diluted with ethyl acetate and washed with water (2×). The organic layer was dried with Na₂SO₄, and concentrated. The crude product was purified by Prep LCLCMS using a gradient of 30%-65% over 15 min to give the title compound (0.010 g, 23% yield).

LCMS m/z: 594.2 (M+H); ¹H-NMR (400 MHz, DMSO) δ 8.13 (d, J=8.0 Hz, 2H), 7.89-7.82 (m, 4H), 7.61-7.58 (m, 2H), 7.52 (s, 1H), 7.35 (dd, J=6.0, 2.0 Hz, 1H), 6.94-6.92 (m, 1H), 6.90-6.88 (m, 1H), 4.26 (d, J=16.0 Hz, 1H), 3.92-3.89 (m, 3H), 3.25 (tt, J=12.0, 4.0 Hz, 1H), 3.17-3.09 (m, 1H), 2.76-2.70 (m, 1H), 2.58 (s, 1H), 1.93-1.88 (m, 2H), 1.48-1.35 (m, 2H).

### Example 39

### 1-[4-(4-{3-[4′-(4,5-Dihydro-1-imidazol-2-yl)-biphenyl-3-yl]-5-methyl-isoxazol-4-yl}-thiazol-2-yl)-piperidin-1-yl]-2-thiophen-2-yl-ethanone

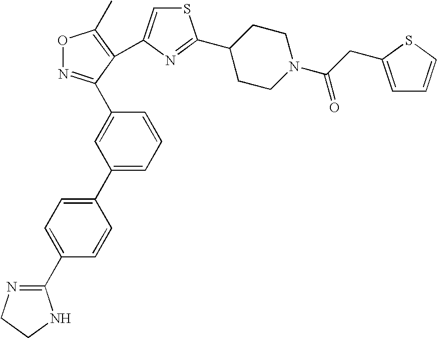

To a dry Smith synthesizer vial was added 3′-(5-Methyl-4-{2-[1-(2-thiophen-2-yl-acetyl)-piperidin-4-yl]-thiazol-4-yl}-isoxazol-3-yl)-biphenyl-4-carbonitrile (0.080 g, 0.146 mmol, see Example 35) and the mixture was sealed under argon atmosphere. Ethylenediamine (0.182 mL, 2.70 mmol) and carbon disulfide (0.009 mL, 1.46×10⁻⁴ mmol) were added and stirred for 2 h at 120° C. The reaction mixture was cooled to ambient temperature and cold water was added. The product was extracted with dichloromethane, dried with Na₂SO₄, and concentrated to give the title compound (0.033 g, 38% yield).

LCMS m/z 594.5 (M+H); ¹H-NMR (400 MHz, DMSO) δ 10.5 (s, 2H), 8.02 (d, J=8.0 Hz, 2H), 7.93-7.89 (m, 3H), 7.86 (s, 1H), 7.63-7.57 (m, 2H), 7.53 (s, 1H), 7.35 (dd, J=10.3 Hz, 1H), 6.93-6.94 (m, 1H), 6.91-6.90 (m, 1H), 4.22 (d, J=12.0 Hz, 1H), 4.02 (s, 4H), 3.94-3.90 (m, 3H), 3.23 (tt, J=11.0, 3.3 Hz, 1H), 3.16-3.11 (m, 1H), 2.74-2.69 (m, 1H), 2.58 (s, 3H), 1.91-1.84 (m, 2H), 1.48-1.27 (m, 2H).

### Example A

### In Vitro Biological Activity

Compounds of the invention were tested for their ability to activate an FSH receptor protein using an AlphaScreen cAMP detection kit (Perkin Elmer; Cat. No. 6760600R).

AV12 cells stably transfected with an expression vector containing an FSH construct and cultured under conditions permissive for cell surface expression of the encoded FSH receptor were harvested from flasks via non-enzymatic means. The cells were washed in PBS and resuspended in Stimulation Buffer. Live cells were counted using a hemacytometer and Trypan blue exclusion, and the cell concentration was adjusted to 2×10⁵ cells/ml. A cAMP standard curve in buffer was prepared with a high final concentration of 1 μM, and serially diluted 1:5 in buffer (10 μL/well). Candidate compounds identified as per above (if frozen, thawed at room temperature) were added to their respective wells (in a 384-well plate) at increasing concentrations (5 μL/well; 10 μM final top assay concentration). To these wells, 2,000 cells in 10 μl of Buffer were added and the mixture as well as 5 μL of cAMP Acceptor Beads in Stimulation Buffer. Following a 30 minute incubation, 5 μL of Donor Bead/Biotinylated cAMP was added to each well, followed by incubation at RT for 2 hours. Plates were read in a Perkin Elmer AlphaQuest™ plate reader per manufacturer instructions.

### Example B

### Biological Activity

The biological in vitro activity of compounds of the invention was determined using the cAMP Whole Cell method as described in Example A. Certain compounds of Examples 8-39 were determined to have an EC50 less than about 200 μM. Activity data for one example compound is provided in the table below.

| | FSH (EC₅₀) |
|---|---|
| Example No. | cAMP Whole Cell (μM) |
| 38 | 1.10 |

* Value is an average of two (2) trials.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

What is claimed is:

1. A compound of Formula I: 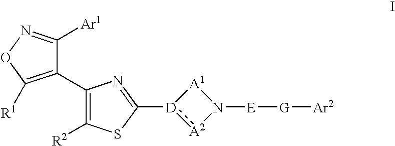
or a pharmaceutically acceptable salt, hydrate or solvate thereof, wherein:
Ar¹ is aryl, heteroaryl, biaryl, biheteroaryl, arylheteroaryl or heteroarylaryl, wherein Ar¹ is optionally substituted with one or more substituents selected from halo, cyano, nitro, C₁₋₆ alkyl, C₁₋₆ haloalkyl, C₂₋₆ alkenyl, C₂₋₆ alkynyl, carbocyclyl optionally substituted by one or more R¹³, heterocyclyl optionally substituted by one or more R¹³, carbocyclylalkyl optionally substituted by one or more R¹³, carbocyclylalkenyl optionally substituted by one or more R¹³, carbocyclylalkynyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸;
Ar² is aryl or heteroaryl, each optionally substituted with one or more substituents selected from halo, cyano, nitro, C₁₋₆ alkyl, C₁₋₆ haloalkyl, C₂₋₆ alkenyl, C₂₋₆ alkynyl, carbocyclyl optionally substituted by one or more R¹⁴, heterocyclyl optionally substituted by one or more R¹⁴, hydroxylamino, OR⁹, SR⁹, SOR¹⁰, SO₂R¹⁰, COR¹⁰, COOR⁹, OC(O)R¹⁰ or NR¹¹R¹²;
D is N, C or CR³; [symbol]
is a single bond when D is N or CR³; [symbol]
is a double bond when D is C;
A¹ is absent or a C₁₋₃ straight-chain aliphatic group optionally substituted with one or more substituents selected from halo, C₁₋₄ alkyl, C₁₋₄ haloalkyl, C₁₋₄ alkoxy, C₁₋₄ haloalkoxy, amino, (C₁₋₆ alkyl)amino, di(C₁₋₆ alkyl)amino, hydroxy, carboxy, (C₁₋₄ alkoxy)carbonyl, or cyano;
A² is C₁₋₄ straight-chain aliphatic group optionally substituted with one or more substituents selected from halo, C₁₋₄ alkyl, C₁₋₄ haloalkyl, C₁₋₄ alkoxy, C₁₋₄ haloalkoxy, amino, (C₁₋₆ alkyl)amino, di(C₁₋₆ alkyl)amino, hydroxy, carboxy, (C₁₋₄ alkoxy)carbonyl, or cyano;
E is CO, C(O)O, C(O)NR⁴, NR⁴CONR⁴, SO, SO₂, SONR⁴, SO₂NR⁴, or a bond;
G is C₁₋₃ alkylene, C₂₋₃ alkenylene or C₂₋₃ alkynylene optionally substituted with one or more substituents selected from halo, C₁₋₄ alkyl, C₁₋₄ haloalkyl, C₁₋₄ alkoxy, C₁₋₄ haloalkoxy, amino, (C₁₋₄ alkyl)amino, di(C₁₋₄ alkyl)amino, hydroxy, carboxy, (C₁₋₄ alkoxy)carbonyl, or cyano;
R¹ is H, C₁₋₆ alkyl, C₂₋₆ alkenyl or C₂₋₆ alkynyl, wherein R¹ is optionally substituted with one or more substituents selected from halo, OH, SH, nitro, cyano, C₁₋₄ haloalkyl, C₁₋₅ acyl, C₁₋₅ acyloxy, C₁₋₄ alkoxy, C₁₋₄ thioalkoxy, C₁₋₄ haloalkoxy, amino, (C₁₋₄ alkyl)amino, di(C₁₋₄ alkyl)amino, aminocarbonyl, (C₁₋₄ alkyl)aminocarbonyl, di(C₁₋₄ alkyl)aminocarbonyl, C₁₋₄ alkylsulfinyl, C₁₋₄ alkylsulfonyl, C₁₋₄ haloalkylsulfinyl, C₁₋₄ haloalkylsulfonyl, aminosulfonyl, (C₁₋₄ alkyl)aminosulfonyl, di(C₁₋₄ alkyl)aminosulfonyl, ureido, C₁₋₄ alkylureido, di(C₁₋₄ alkyl)ureido, thioureido, C₁₋₄ alkylthioureido, di(C₁₋₄ alkyl)thioureido, carboxy, (C₁₋₆ alkoxy)carbonyl, and hydroxylamino;
R² is H, C₁₋₆ alkyl, C₂₋₆ alkenyl or C₂₋₆ alkynyl, wherein R² is optionally substituted with one or more substituents selected from halo, OH, SH, nitro, cyano, C₁₋₄ haloalkyl, C₁₋₅ acyl, C₁₋₅ acyloxy, C₁₋₄ alkoxy, C₁₋₄ thioalkoxy, C₁₋₄ haloalkoxy, amino, (C₁₋₄ alkyl)amino, di(C₁₋₄ alkyl)amino, aminocarbonyl, (C₁₋₄ alkyl)aminocarbonyl, di(C₁₋₄ alkyl)aminocarbonyl, C₁₋₄ alkylsulfinyl, C₁₋₄ alkylsulfonyl, C₁₋₄ haloalkylsulfinyl, C₁₋₄ haloalkylsulfonyl, aminosulfonyl, (C₁₋₄ alkyl)aminosulfonyl, di(C₁₋₄ alkyl)aminosulfonyl, ureido, C₁₋₄ alkylureido, di(C₁₋₄ alkyl)ureido, thioureido, C₁₋₄ alkylthioureido, di(C₁₋₄ alkyl)thioureido, carboxy, (C₁₋₆ alkoxy)carbonyl, and hydroxylamino;
or R¹ and R² together with the carbon atoms to which they are attached and the two carbon atoms through which the isoxazole and thiazole moieties of the core are joined form a fused C₅₋₇ carbocyclyl group or fused 5-7 membered heterocyclyl group optionally substituted with one or more substituents selected from halo, C₁₋₄ alkyl, C₁₋₄ haloalkyl, C₁₋₄ alkoxy, C₁₋₄ haloalkoxy, amino, (C₁₋₄ alkyl)amino, di(C₁₋₄ alkyl)amino, hydroxy, carboxy, (C₁₋₄ alkoxy)carbonyl, or cyano;
R³ is H or C₁₋₆ alkyl;
R⁴, at each independent occurrence, is H or C₁₋₄ alkyl;
R⁵ and R⁹ are each, independently, H, C₁₋₈ alkyl, C₁₋₈ haloalkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl or (5-7 membered heterocycloalkyl)alkyl;
R⁶ and R¹⁰ are each, independently, H, C₁₋₈ alkyl, C₁₋₈ haloalkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, amino, (C₁₋₄ alkyl)amino, di(C₁₋₄ alkyl)amino,
R⁷ and R⁸ are each, independently, H, C₁₋₈ alkyl, C₂₋₈ alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, (C₁₋₈ alkyl)carbonyl, (C₁₋₈ haloalkyl)carbonyl, (C₁₋₈ alkoxy)carbonyl, (C₁₋₈ haloalkoxy)carbonyl, (C₁₋₄ alkyl)sulfonyl, (C₁₋₄ haloalkyl)sulfonyl or arylsulfonyl;
or R⁷ and R⁸, together with the N atom to which they are attached form a 5-7 membered heterocycloalkyl group;
R¹¹ and R¹² are each, independently, H, C₁₋₈ alkyl, C2-g alkenyl, C₂₋₈ alkynyl, aryl, heteroaryl, C₃₋₇ cycloalkyl, 5-7 membered heterocycloalkyl, arylalkyl, heteroarylalkyl, (C₃₋₇ cycloalkyl)alkyl, (5-7 membered heterocycloalkyl)alkyl, (C₁₋₈ alkyl)carbonyl, (C₁₋₈ haloalkyl)carbonyl, (C₁₋₈ alkoxy)carbonyl, (C₁₋₈ haloalkoxy)carbonyl, (C₁₋₄ alkyl)sulfonyl, (C₁₋₄ haloalkyl)sulfonyl or arylsulfonyl;
or R¹¹ and R¹², together with the N atom to which they are attached form a 5-7 membered heterocycloalkyl group; and
R¹³ and R¹⁴ are each, independently, halo, cyano, nitro, C₁₋₄ alkyl, C₁₋₄ haloalkyl, C₁₋₄ alkoxy, C₁₋₄ haloalkoxy, amino, (C₁₋₄ alkyl)amino, di(C₁₋₄ alkyl)amino, hydroxy, carboxy, (C₁₋₄ alkoxy)carbonyl, C₁₋₄ acyl, C₁₋₄ acyloxy, aminocarbonyl, (C₁₋₄ alkyl)aminocarbonyl, or di(C₁₋₄ alkyl)aminocarbonyl.

2. The compound of claim 1 wherein Ar¹ is aryl, heteroaryl, biaryl, biheteroaryl, arylheteroaryl or heteroarylaryl, wherein Ar¹ is substituted with 1, 2 or 3 substituents selected from halo, cyano, nitro, C₁₋₆ alkyl, C₁₋₆ haloalkyl, C₂₋₆ alkenyl, C₂₋₆ alkynyl, carbocyclyl optionally substituted by one or more R¹³, heterocyclyl optionally substituted by one or more R¹³, carbocyclylalkyl optionally substituted by one or more R¹³, carbocyclylalkenyl optionally substituted by one or more R¹³, carbocyclylalkynyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸.

3. The compound of claim 1 wherein Ar¹ is aryl, heteroaryl, biaryl, biheteroaryl, arylheteroaryl or heteroarylaryl, wherein Ar¹ is optionally substituted with one or more substituents selected from halo, cyano, nitro, heterocyclyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸.

4. The compound of claim 1 wherein Ar¹ is aryl, biaryl or heteroarylaryl, wherein Ar¹ is optionally substituted with one or more substituents selected from halo, cyano, nitro, C₁₋₆ alkyl, C₁₋₆ haloalkyl, C₂₋₆ alkenyl, C₂₋₆ alkynyl, carbocyclyl optionally substituted by one or more R¹³, heterocyclyl optionally substituted by one or more R¹³, carbocyclylalkyl optionally substituted by one or more R¹³, carbocyclylalkenyl optionally substituted by one or more R¹³, carbocyclylalkynyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸.

5. The compound of claim 1 wherein Ar¹ is phenyl, biphenyl or heteroarylphenyl, wherein Ar¹ is optionally substituted with one or more substituents selected from halo, cyano, nitro, C₁-C₆ alkyl, C₁-C₆ haloalkyl, C₂-C₆ alkenyl, C₂-C₆ alkynyl, carbocyclyl optionally substituted by one or more R¹³, heterocyclyl optionally substituted by one or more R¹³, carbocyclylalkyl optionally substituted by one or more R¹³, carbocyclylalkenyl optionally substituted by one or more R¹³, carbocyclylalkynyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸.

6. The compound of claim 1 wherein Ar¹ is phenyl, biphenyl or heteroarylphenyl, wherein Ar¹ is optionally substituted with 1, 2 or 3 substituents selected from halo, cyano, nitro, heterocyclyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸.

7. The compound of claim 1 wherein Ar¹ is phenyl, biphenyl or heteroarylphenyl, wherein Ar¹ is substituted with 1, 2 or 3 substituents selected from halo, cyano, nitro, heterocyclyl optionally substituted by one or more R¹³, heterocyclylalkynyl optionally substituted by one or more R¹³, C₁₋₄ alkoxy, SO₂R⁶, COR⁶, COOR⁵ or NR⁷R⁸.

8. The compound of claim 1 wherein Ar² is aryl or heteroaryl, each optionally substituted with 1, 2 or 3 substituents selected from halo, cyano, nitro, C₁₋₆ alkyl, C₁₋₆ haloalkyl, C₂₋₆ alkenyl, C₂₋₆ alkynyl, carbocyclyl optionally substituted by one or more R¹⁴, heterocyclyl optionally substituted by one or more R¹⁴, hydroxylamino, OR⁹, SR⁹, SOR¹⁰, SO₂R¹⁰, COR¹⁰, COOR⁹, OC(O)R¹⁰ or NR¹¹R¹².

9. The compound of claim 1 wherein Ar² is aryl or heteroaryl.

10. The compound of claim 1 wherein Ar² is heteroaryl.

11. The compound of claim 1 wherein Ar² is thienyl.

12. The compound of claim 1 wherein Ar² is aryl.

13. The compound of claim 1 wherein Ar² is phenyl.

14. The compound of claim 1 wherein D is CR³.

15. The compound of claim 1 wherein D is CH.

16. The compound of claim 1 wherein A¹ is a C₁₋₃ alkylene group.

17. The compound of claim 1 wherein A¹ is CH₂ or CH₂CH₂.

18. The compound of claim 1 wherein A¹ is absent.

19. The compound of claim 1 wherein D is CR³ and A² is a C₁₋₃ alkylene group.

20. The compound of claim 1 wherein D is CR³ and A² is CH₂CH₂ or CH₂CH₂CH₂.

21. The compound of claim 1 wherein D is CR³, A¹ is CH₂CH₂, and A² is CH₂CH₂.

22. The compound of claim 1 wherein D is CR³, A¹ is absent, and A² is CH₂CH₂CH₂.

23. The compound of claim 1 wherein E is CO, C(O)O, C(O)NR⁴, SO₂ or a bond.

24. The compound of claim 1 wherein E is CO or SO₂.

25. The compound of claim 1 wherein E is CO.

26. The compound of claim 1 wherein G is C₁₋₃ alkylene.

27. The compound of claim 1 wherein G is CH₂ or CH₂CH₂.

28. The compound of claim 1 wherein G is CH₂.

29. The compound of claim 1 wherein R¹ is H or C₁₋₄ alkyl.

30. The compound of claim 1 wherein R¹ is methyl.

31. The compound of claim 1 wherein:
R¹ is H, C₁₋₆ alkyl, C₂₋₆ alkenyl or C₂₋₆ alkynyl, wherein R¹ is optionally substituted with one or more substituents selected from halo, OH, SH, nitro, cyano, C₁₋₄ haloalkyl, C₁₋₅ acyl, C₁₋₅ acyloxy, C₁₋₄ alkoxy, C₁₋₄ thioalkoxy, C₁₋₄ haloalkoxy, amino, (C₁₋₄ alkyl)amino, di(C₁₋₄ alkyl)amino, aminocarbonyl, (C₁₋₄ alkyl)aminocarbonyl, di(C₁₋₄ alkyl)aminocarbonyl, C₁₋₄ alkylsulfinyl, C₁₋₄ alkylsulfonyl, C₁₋₄ haloalkylsulfinyl, C₁₋₄ haloalkylsulfonyl, aminosulfonyl, (C₁₋₄ alkyl)aminosulfonyl, di(C₁₋₄ alkyl)aminosulfonyl, ureido, C₁₋₄ alkylureido, di(C₁₋₄ alkyl)ureido, thioureido, C₁₋₄ alkylthioureido, di(C₁₋₄ alkyl)thioureido, carboxy, (C₁₋₆ alkoxy)carbonyl, and hydroxylamino; and
R² is H, C₁₋₆ alkyl, C₂₋₆ alkenyl or C₂₋₆ alkynyl, wherein R² is optionally substituted with one or more substituents selected from halo, OH, SH, nitro, cyano, C₁₋₄ haloalkyl, C₁₋₅ acyl, C₁₋₅ acyloxy, C₁₋₄ alkoxy, C₁₋₄ thioalkoxy, C₁₋₄ haloalkoxy, amino, (C₁₋₄ alkyl)amino, di(C₁₋₄ alkyl)amino, aminocarbonyl, (C₁₋₄ alkyl)aminocarbonyl, di(C₁₋₄ alkyl)aminocarbonyl, C₁₋₄ alkylsulfinyl, C₁₋₄ alkylsulfonyl, C₁₋₄ haloalkylsulfinyl, C₁₋₄ haloalkylsulfonyl, aminosulfonyl, (C₁₋₄ alkyl)aminosulfonyl, di(C₁₋₄ alkyl)aminosulfonyl, ureido, C₁₋₄ alkylureido, di(C₁₋₄ alkyl)ureido, thioureido, C₁₋₄ alkylthioureido, di(C₁₋₄ alkyl)thioureido, carboxy, (C₁₋₆ alkoxy)carbonyl, and hydroxylamino.

32. The compound of claim 1 wherein R² is H or C₁₋₄ alkyl.

33. The compound of claim 1 wherein R² is H.

34. The compound of claim 1 wherein R³ is H.

35. The compound of claim 1 wherein R⁴, at each independent occurrence, is H.

36. The compound of claim 1 wherein:
D is CR³;
A¹ is a absent or a C₁₋₃ alkylene group;
A² is a C₁₋₃ alkylene group;
E is CO, C(O)O, C(O)NR⁴, SO₂ or a bond;
G is C₁₋₃ alkylene;
R¹ is H or C₁₋₆ alkyl; and
R² is H or C₁₋₆ alkyl.

37. The compound of claim 1 wherein:
Ar² is aryl or heteroaryl, each optionally substituted with 1, 2 or 3 substituents selected from halo, cyano, nitro, C₁-C₆ alkyl, C₁-C₆ haloalkyl, C₂-C₆ alkenyl, C₂-C₆ alkynyl, carbocyclyl optionally substituted by one or more R¹⁴, heterocyclyl optionally substituted by one or more R¹⁴, hydroxylamino, OR⁹, SR⁹, SOR¹⁰, SO₂R¹⁰, COR¹⁰, COOR⁹, OC(O)R¹⁰ or NR¹¹R¹²;
D is CR³;
A¹ is absent or a C₁₋₃ alkylene group;
A² is a C₁₋₃ alkylene group;
E is CO, C(O)O, C(O)NR⁴, SO₂ or a bond;
G is C₁₋₃ alkylene;
R¹ is H or C₁₋₆ alkyl; and
R² is H or C₁₋₆ alkyl.

38. The compound of claim 1 wherein:
Ar¹ is phenyl, biphenyl or heteroarylphenyl, wherein Ar¹ is optionally substituted with one or more substituents selected from halo, cyano, nitro, C₁-C₆ alkyl, C₁-C₆ haloalkyl, C₂-C₆ alkenyl, C₂-C₆ alkynyl, carbocyclyl optionally substituted by one or more R¹³, heterocyclyl optionally substituted by one or more R¹³, carbocyclylalkyl optionally substituted by one or more R¹³, carbocyclylalkenyl optionally substituted by one or more R¹³, carbocyclylalkynyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸;
Ar² is aryl or heteroaryl;
D is CR³;
A¹ is absent or a C₁₋₃ alkylene group;
A² is a C₁₋₃ alkylene group;
E is CO, C(O)O, C(O)NR⁴, SO₂ or a bond;
G is C₁₋₃ alkylene;
R¹ is H or C₁₋₆ alkyl; and
R² is H or C₁₋₆ alkyl.

39. The compound of claim 1 wherein:
Ar¹ is phenyl, biphenyl or heteroarylphenyl, wherein Ar¹ is optionally substituted with 1, 2 or 3 substituents selected from halo, cyano, nitro, heterocyclyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸;
Ar² is aryl or heteroaryl;
D is CR³;
A¹ is absent or a C₁₋₃ alkylene group;
A² is a C₁₋₃ alkylene group;
E is CO, C(O)O, C(O)NR⁴, SO₂ or a bond;
G is C₁₋₃ alkylene;
R¹ is H or C₁₋₆ alkyl; and
R² is H or C₁₋₆ alkyl.

40. The compound of claim 1 wherein:
Ar¹ is phenyl, biphenyl or heteroarylphenyl, wherein Ar¹ is optionally substituted with 1, 2 or 3 substituents selected from halo, cyano, nitro, heterocyclyl optionally substituted by one or more R¹³, heterocyclylalkyl optionally substituted by one or more R¹³, heterocyclylalkenyl optionally substituted by one or more R¹³, heterocyclylalkynyl optionally substituted by one or more R¹³, hydroxylamino, OR⁵, SR⁵, SOR⁶, SO₂R⁶, COR⁶, COOR⁵, OC(O)R⁶ or NR⁷R⁸;
Ar² is aryl or heteroaryl;
D is CH;
A¹ is absent, CH₂ or CH₂CH₂;
A² is CH₂CH₂ or CH₂CH₂CH₂;
E is CO, SO₂ or a bond;
G is CH₂ or CH₂CH₂;
R¹ is C₁₋₄ alkyl; and
R² is H.

41. The compound of claim 1 wherein:
Ar¹ is phenyl, biphenyl or heteroarylphenyl, wherein Ar¹ is substituted with 1, 2 or 3 substituents selected from halo, cyano, nitro, heterocyclyl optionally substituted by one or more R¹³, heterocyclylalkynyl optionally substituted by one or more R¹³, C₁₋₄ alkoxy, SO₂R⁶, COR⁶, COOR⁵ or NR⁷R⁸;
Ar² is aryl or heteroaryl;
D is CH;
A¹ is absent, CH₂ or CH₂CH₂;
A² is CH₂CH₂ or CH₂CH₂CH₂;
E is CO, SO₂ or a bond;
G is CH₂ or CH₂CH₂;
R¹ is C₁₋₄ alkyl; and
R² is H.

42. The compound of claim 1 selected from:
4-{4-[3-(3-Bromo-phenyl)-5-methyl-isoxazol-4-yl]-thiazol-2-yl}-1-phenylmethane-sulfonyl-piperidine;
1-(4-{4-[3-(3-Bromo-phenyl)-5-methyl-isoxazol-4-yl]-thiazol-2-yl}-piperidin-1-yl)-2-thiophen-2-yl-ethanone;
1-(4-{4-[3-(3-Bromo-phenyl)-5-methyl-isoxazol-4-yl]-thiazol-2-yl}-piperidin-1-yl)-2-thiophen-3-yl-ethanone;
1-(2-{4-[3-(3-Bromo-phenyl)-5-methyl-isoxazol-4-yl]-thiazol-2-yl}-pyrrolidin-1-yl)-2-thiophen-2-yl-ethanone;
1-(4-{4-[3-(3-Isopropylamino-phenyl)-5-methyl-isoxazol-4-yl]-thiazol-2-yl}-piperidin-1-yl)-2-thiophen-2-yl-ethanone;
1-[4-(4-{5-Methyl-3-[3-(2-morpholin-4-yl-ethylamino)-phenyl]-isoxazol-4-yl}-thiazol-2-yl)-piperidin-1-yl]-2-thiophen-2-yl-ethanone;
1-(4-{4-[5-Methyl-3-(3-morpholin-4-yl-phenyl)-isoxazol-4-yl]-thiazol-2-yl}-piperidin-1-yl)-2-thiophen-2-yl-ethanone;
1-[4-(4-{5-Methyl-3-[3-(4-methyl-piperazin-1-yl)-phenyl]-isoxazol-4-yl}-thiazol-2-yl)-piperidin-1-yl]-2-thiophen-2-yl-ethanone;
N-[3′-(5-Methyl-4-{2-[1-(2-thiophen-2-yl-acetyl)-piperidin-4-yl]-thiazol-4-yl}-isoxazol-3-yl)-biphenyl-4-yl]-acetamide;
N-[3′-(5-Methyl-4-{2-[1-(2-thiophen-2-yl-acetyl)-piperidin-4-yl]-thiazol-4-yl}-isoxazol-3-yl)-biphenyl-3-yl]-acetamide;
3′-(5-Methyl-4-{2-[1-(2-thiophen-2-yl-acetyl)-piperidin-4-yl]-thiazol-4-yl}-isoxazol-3-yl)-biphenyl-4-carboxylic acid amide;
3′-(5-Methyl-4-{2-[1-(2-thiophen-2-yl-acetyl)-piperidin-4-yl]-thiazol-4-yl}-isoxazol-3-yl)-biphenyl-3-carboxylic acid amide;
3′-{5-Methyl-4-[2-(1-phenylmethanesulfonyl-piperidin-4-yl)-thiazol-4-yl]-isoxazol-3-yl}-biphenyl-4-carboxylic acid;
3′-(5-Methyl-4-{2-[1-(2-thiophen-2-yl-acetyl)-piperidin-4-yl]-thiazol-4-yl}-isoxazol-3-yl)-biphenyl-4-carboxylic acid;
3′-(5-Methyl-4-{2-[1-(2-thiophen-2-yl-acetyl)-pyrrolidin-2-yl]-thiazol-4-yl}-isoxazol-3-yl)-biphenyl-4-carboxylic acid;
3′-(5-Methyl-4-{2-[1-(2-thiophen-2-yl-acetyl)-piperidin-4-yl]-thiazol-4-yl}-isoxazol-3-yl)-biphenyl-3-carboxylic acid;
1-(4-{4-[5-Methyl-3-(3-pyridin-4-yl-phenyl)-isoxazol-4-yl]-thiazol-2-yl}-piperidin-1-yl)-2-thiophen-2-yl-ethanone;
1-(4-{4-[5-Methyl-3-(3-pyrimidin-5-yl-phenyl)isoxazol-4-yl]-thiazol-2-yl}-piperidin-1-yl)-2-thiophen-2-yl-ethanone;
1-(4-{4-[3-(4′-Methoxy-biphenyl-3-yl)-5-methyl-isoxazol-4-yl]-thiazol-2-yl}-piperidin-1-yl)-2-thiophen-2-yl-ethanone;
3′-(5-Methyl-4-{2-[1-(2-thiophen-2-yl-acetyl)-piperidin-4-yl]-thiazol-4-yl}-isoxazol-3-yl)-biphenyl-4-carboxylic acid dimethylamide;
3′-(5-Methyl-4-{2-[1-(2-thiophen-2-yl-acetyl)-piperidin-4-yl]-thiazol-4-yl}-isoxazol-3-yl)-biphenyl-4-carboxylic acid methylamide;
1-[4-(4-{5-Methyl-3-[4′-(morpholine-4-carbonyl)-biphenyl-3-yl]-isoxazol-4-yl}-thiazol-2-yl)-piperidin-1-yl]-2-thiophen-2-yl-ethanone;
1-[4-(4-{5-Methyl-3-[3′-(morpholine-4-carbonyl)-biphenyl-3-yl]-isoxazol-4-yl}-thiazol-2-yl)-piperidin-1-yl]-2-thiophen-2-yl-ethanone;
1-(4-{4-[3-(4′-Amino-biphenyl-3-yl)-5-methyl-isoxazol-4-yl]-thiazol-2-yl}-piperidin-1-yl)-2-thiophen-2-yl-ethanone;
N-[3′-(5-Methyl-4-{2-[1-(2-thiophen-2-yl-acetyl)-piperidin-4-yl]-thiazol-4-yl}-isoxazol-3-yl)-biphenyl-4-yl]-methanesulfonamide;
1-(4-{4-[3-(4′-Methanesulfonyl-biphenyl-3-yl)-5-methyl-isoxazol-4-yl]-thiazol-2-yl}-piperidin-1-yl)-2-thiophen-2-yl-ethanone;
2,2,2-Trifluoro-N-[3′-(5-methyl-4-{2-[1-(2-thiophen-2-yl-acetyl)-piperidin-4-yl]-thiazol-4-yl}-isoxazol-3-yl)-biphenyl-3-yl]-acetamide;
1-(4-{4-[3-(3′-Methanesulfonyl-biphenyl-3-yl)-5-methyl-isoxazol-4-yl]-thiazol-2-yl}-piperidin-1-yl)-2-thiophen-2-yl-ethanone;
3′-(5-Methyl-4-{2-[1-(2-thiophen-2-yl-acetyl)-piperidin-4-yl]-thiazol-4-yl}-isoxazol-3-yl)-biphenyl-4-carbonitrile;
1-{4-[4-(3-{3-[3-(1,1-Dioxo-1λ⁶-thiomorpholin-4-yl)-prop-1-ynyl]-phenyl}-5-methyl-isoxazol-4-yl)-thiazol-2-yl]-piperidin-1-yl}-2-thiophen-2-yl-ethanone;
1-[4-(4-{5-Methyl-3-[4′-(1H-tetrazol-5-yl)-biphenyl-3-yl]-isoxazol-4-yl}-thiazol-2-yl)-piperidin-1-yl]-2-thiophen-2-yl-ethanone; and
1-[4-(4-{3-[4′-(4,5-Dihydro-1H-imidazol-2-yl)-biphenyl-3-yl]-5-methyl-isoxazol-4-yl}-thiazol-2-yl)-piperidin-1-yl]-2-thiophen-2-yl-ethanone;
or pharmaceutically acceptable salt thereof.

43. A composition comprising a compound of claim 1 and a pharmaceutically acceptable carrier.

44. A method of modulating the follicle stimulating hormone (FSH) receptor comprising contacting said receptor with a compound of claim 1.

45. A method of activating the follicle stimulating hormone (FSH) receptor comprising contacting said receptor with a compound of claim 1.

46. A method of increasing the adenylyl cyclase activity or the level of 5′-monophosphate (cAMP) in a cell, cell culture or tissue expressing the follicle stimulating hormone receptor comprising contacting said cell, cell culture or tissue with a compound of claim 1.

47. A method of inducing ovulation in a female mammal comprising administering to said female mammal an ovulation-inducing amount of a compound of claim 1.

48. A method of treating a fertility disorder in a patient comprising administering to said patient a therapeutically effective amount of a compound of claim 1.

49. A method of treating infertility in a female patient comprising administering to said female patient a therapeutically effective amount of a compound of claim 1.

50. A compound according to any one of claims 1 to 42 for use in therapy.

51. A compound according to any one of claims 1 to 42 for use in the treatment of a fertility disorder in a patient.

52. A compound according to any one of claims 1 to 42 for use in the treatment of infertility in a female patient.

53. A compound according to any one of claims 1 to 42 for use in the preparation of a medicament for use in therapy.

54. A compound according to any one of claims 1 to 42 for use in the preparation of a medicament for use in the treatment of a fertility disorder in a patient.

55. A compound according to any one of claims 1 to 42 for use in the preparation of a medicament for use in the treatment of infertility in a female patient.

56. Use of a compound according to any one of claims 1 to 42 for the manufacture of a medicament.

57. Use of a compound according to any one of claims 1 to 42 for the manufacture of a medicament for the treatment of a fertility disorder in a patient.

58. Use of a compound according to any one of claims 1 to 42 for the manufacture of a medicament for the treatment of infertility in a female patient.
